(19) United States
(12) Patent Application Publication
(10) Pub. No.: US 2024/0130678 A1
(43) Pub. Date: Apr. 25, 2024

(54) TEXTILE CONFIGURED FOR STRAIN SENSING, METHOD OF MANUFACTURING A TEXTILE FOR STRAIN SENSING AND A KNITTING APPARATUS THEREOF

(71) Applicant: SINGAPORE UNIVERSITY OF TECHNOLOGY AND DESIGN, Singapore (SG)

(72) Inventors: Hong Yee LOW, Singapore (SG); Ujjaval GUPTA, Singapore (SG); Jun Liang LAU, Singapore (SG); Gim Song SOH, Singapore (SG); Pei Zhi CHIA, Singapore (SG); Alvee AHMED, Singapore (SG)

(73) Assignee: SINGAPORE UNIVERSITY OF TECHNOLOGY AND DESIGN, Singapore (SG)

(21) Appl. No.: 18/546,368

(22) PCT Filed: Feb. 25, 2022
(86) PCT No.: PCT/SG2022/050092

§ 371 Date: Aug. 14, 2023

(30) Foreign Application Priority Data
Feb. 25, 2021 (SG) 10202101960Y
Oct. 1, 2021 (SG) 10202111004S

(51) Int. Cl. A61B 5/00; D04B 1/10; D04B 1/16; D04B 1/24

(52) U.S. Cl. CPC A61B 5/6804; D04B 1/102; D04B 1/16; D04B 1/24; A61B 2562/0261; D10B 2331/02; D10B 2401/16; D10B 2403/02431; D10B 2509/00

(57) ABSTRACT

There is provided a textile including a strain sensing portion integrally knitted therein and including groups of courses of yarn, each group including a first course of conductive yarn, a second course of dielectric yarn, and a third course of conductive yarn, each course including a plurality of knitted loops. The second course is arranged in between the first and third courses and configured to have a higher elasticity than the first and third courses such that when the strain sensing portion is at a relaxed state, corresponding knitted loops of the first and third courses are caused by a corresponding knitted loop of the second course to contact each other, and when the strain sensing portion is at a stretched state, corresponding knitted loops of the first and third courses are caused by a corresponding knitted loop of the second course to be not in contact with each other.

### CROSS-REFERENCE TO RELATED APPLICATION

This application claims the benefit of priority of Singapore Patent Application No. 10202101960Y, filed on 25 Feb. 2021, and Singapore Patent Application No. 10202111004S, filed on 1 Oct. 2021, the content of which being hereby incorporated by reference in their entirety for all purposes.

### TECHNICAL FIELD

The present invention generally relates to a textile configured for strain sensing, a method of manufacturing a textile for strain sensing and a knitting apparatus for manufacturing a textile for strain sensing.

### BACKGROUND

Long-term continuous monitoring is vital to track various healthcare conditions. This is enabled by a range of wearables that are commercially available today. However, most of these wearable devices are made from rigid materials such as metals and plastics which are bulky and have limited conformability to human body parts. An alternative product that overcomes these limitations would be e-textiles that have been used for the development of soft wearable devices.

E-textiles used for the development of soft wearable devices may have stretchability, breathability, low self-weight and soft hand feel, and serve as an excellent material choice for the development of soft robotics and soft sensors that can be integrated into daily knitwear.

A textile-based wearable has several key components: (a) the sensors to measure specific parameters from the human body, and (b) the network of stretchable electrical systems that can carry signals to-and-fro. In the literature, some of these fabric-based wearables have been proposed for respiratory monitoring, joint motion sensing, etc. In most of these works, the sensing component is either made of commercially available conductive fabric that is manually sewn to the wearer's external garment or made by manual drop casting methods, thus greatly limiting their scalability in terms of fabrication. Besides, their sensitivity is often limited by the overlying homogeneous patch of commercially available conductive fabric. Additionally, there has been limited work addressing the need for a network of stretchable electrical systems. At present, the existing literature documents only a few examples of integrated interconnects and circuitry components within a garment using embroidery or sewing techniques. However significant, these approaches provide minimal control over the substrate shape and properties (stretchability, breathability etc.), which is often limited by the underlying garment.

A need therefore exists to provide a textile configured for strain sensing that seeks to overcome, or at least ameliorate, one or more of the deficiencies of conventional textiles configured for strain sensing, and more particularly, providing a textile configured for strain sensing which is more effective in strain sensing (e.g., more sensitive and having a larger working range). It is against this background that the present invention has been developed.

### SUMMARY

According to a first aspect of the present invention, there is provided a textile configured for strain sensing comprising:
a strain sensing portion integrally knitted in the textile, wherein the strain sensing portion comprises:
a plurality of first groups of courses of yarn, each first group of courses of yarn comprising:
   a first course of conductive yarn comprising a plurality of knitted loops;
   a second course of dielectric yarn comprising a plurality of knitted loops; and
   a third course of conductive yarn comprising a plurality of knitted loops,
   wherein the second course is arranged in between the first and third courses and configured to have a higher elasticity than the first and third courses such that:
      when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and
      when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

According to a second aspect of the present invention, there is provided a method of manufacturing a textile for strain sensing, the method comprising:
forming a strain sensing portion integrally knitted in the textile, wherein forming the strain sensing portion comprises:
knitting a plurality of first groups of courses of yarn, each first group of courses of yarn comprising:
a first course of conductive yarn comprising a plurality of knitted loops;
a second course of dielectric yarn comprising a plurality of knitted loops; and
a third course of conductive yarn comprising a plurality of knitted loops,
wherein the second course is arranged in between the first and third courses and configured to have a higher elasticity than the first and third courses such that:
   when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and
   when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

According to third aspect of the present invention, there is provided a knitting apparatus for manufacturing a textile for strain sensing, the knitting apparatus comprising:
a needle controller configured to control one or more needles for performing knitting;
a memory; and
at least one processor communicatively coupled to the memory and the needle controller, and configured to perform a method of manufacturing a textile configured for strain sensing according to the above-mentioned first aspect of the present invention.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic drawing of a textile configured for strain sensing according to various embodiments of the present invention;

FIG. 2 depicts a schematic flow diagram of a method of manufacturing a textile for strain sensing according to various embodiments;

FIG. 3 depicts a schematic block diagram of a knitting apparatus for manufacturing a textile for strain sensing, such as the textile configured for strain sensing as described herein with reference to FIG. 1 according to various embodiments;

FIG. 4A shows a schematic of a knitted loop according to various example embodiments;

FIG. 4B shows various contact points in a stitch pattern according to various example embodiments;

FIG. 5 shows basic stitch types forming a conductive loop, according to various example embodiments;

FIG. 6 shows a circuit diagram of the soft wearable knee brace showing resistor and sensor connected via interconnects, according to various example embodiments;

FIG. 7A shows a unit cell of the knit instruction according to various example embodiments;

FIG. 7B shows a graphical simulation of the knitted loops for a unit cell containing alternate courses of conductive and non-conductive yarn, according to various example embodiments;

FIGS. 8A-8B show the schematic of the knitted loops in the conductive knit during knitting and at rest state after knitting, respectively, according to various example embodiments;

FIG. 9A shows a graph of voltage vs strain response of the alternate conductive and non-conductive knit, according to various example embodiments;

FIGS. 9B-9D show microscopic images of the alternate conductive and non-conductive knit at rest state, 100% strain and 125% strain, respectively, according to various example embodiments;

FIGS. 9A-9B show generic flight path of square cyclic control for directional control according to various example embodiments;

FIG. 10 shows the comparison of the sensor's performance among conductive knits with different yarn material combinations, according to various example embodiments;

FIG. 11A shows the unit cell which has conductive knit stitches separated by miss stitches, according to various example embodiments, and FIG. 11B shows a graphical simulation of the stitch pattern in conductive knit, according to various example embodiments;

FIG. 12A shows a schematic of the course-wise resistors with knit-miss stitch, according to various example embodiments; and FIGS. 12B-12E show microscope images of several courses of the resistor stitch pattern, with each resistor course insulated from the adjacent resistor courses by courses knitted with non-conductive yarn, according to various example embodiments;

FIG. 13 shows the voltage vs strain graph of the course wise knitted resistor, according to various example embodiments;

FIG. 14A shows another unit cell of knit instruction and FIG. 14B shows a graphical simulation of the knitted loops with the unit cell, according to various example embodiments;

FIG. 15A shows a schematic of course wise interconnects, and FIGS. 15B-15C show microscopic images of the course wise interconnects at rest state and 50% strain, respectively, according to various example embodiments;

FIG. 16 shows the voltage vs strain graph for the course wise knitted interconnect, according to various example embodiments;

FIG. 17A shows the unit cell for a wale wise interconnect, and FIG. 17B shows a graphical simulation of the stitch pattern in conductive knit, according to various example embodiments;

FIG. 18A shows another schematic of a unit cell of the wale wise interconnects displaying the contact points, FIG. 18B shows the conductive loops in a wale wise knitted interconnect at rest state, FIG. 18C and FIG. 18D show the loops when the interconnect is stretched by 50% and 75% strain, respectively, according to various example embodiments;

FIG. 19 shows a voltage vs strain graph for the wale wise knitted interconnect, according to various example embodiments;

FIGS. 20A-20B show a left side view, and a right side view of a knee brace, showing the distribution of the sensor, interconnects and resistor in a wearable according to various example embodiments;

FIG. 21 shows an all knitted soft wearable knee brace with all the electrical components integrated as a single holistic device according to various example embodiments;

FIG. 22 shows an image illustrating a knitted conductive sample mounted on a linear cyclic strain test setup and close-up diagrams of the knitted loops, according to various example embodiments;

FIG. 23 shows a voltage-strain graph for one Rib 1×1 sample showing how the working range is determined for a single loading cycle, according to various example embodiments;

FIG. 24 depicts a Table I showing the means and standard deviations of the gauge factor, working range, and resistance measurements for each stitch pattern, according to various example embodiments;

FIG. 25 depicts a Table II showing results of comparison of pairs of group means, according to various example embodiments;

FIGS. 26A-26C show schematics of a wale wise knitted sensor, single jersey stitch pattern of the conductive fabric, and purl stitch pattern of the surrounding fabric, respectively, according to various example embodiments;

FIG. 27 shows the effect of loop length on gauge factor and working range of the sensors, according to various example embodiments;

FIG. 28 shows the knitted sensors of various stitch patterns, according to various example embodiments;

FIG. 29A shows the stitch pattern of a single jersey knitted sensor, according to various example embodiments;

FIG. 29B shows the stitch pattern of a purl knitted sensor, according to various example embodiments;

FIG. 29C shows the stitch pattern of a full cardigan knitted sensor, according to various example embodiments;

FIG. 29D shows the stitch pattern of a half cardigan knitted sensor, according to various example embodiments;

FIG. 29E shows the stitch pattern of a mesh stitches knitted sensor, according to various example embodiments;

FIG. 29F shows the stitch pattern of a Pique lacoste stitches knitted sensor, according to various example embodiments;

FIG. 29G shows the normalized voltage vs strain response of the single jersey knitted sensor of FIG. 29A, according to various example embodiments;

FIG. 29H shows the normalized voltage vs strain response of the knitted sensor of FIG. 29B, according to various example embodiments;

FIG. 29I shows the normalized voltage vs strain response of the knitted sensor of FIG. 29C, according to various example embodiments;

FIG. 29J shows the normalized voltage vs strain response of the knitted sensor of FIG. 29D, according to various example embodiments;

FIG. 29K shows the normalized voltage vs strain response of the knitted sensor of FIG. 29E, according to various example embodiments;

FIG. 29L shows the normalized voltage vs strain response of the knitted sensor of FIG. 29F, according to various example embodiments;

FIG. 30 shows the comparison of the gauge factor and working range of the sensors knitted with various stitch patterns, according to various example embodiments;

FIGS. 31A-31B show images of the design of all conductive courses sensor and design of alternate conductive courses sensor, respectively;

FIG. 31C show a graph illustrating a comparison of performance between all conductive and alternate conductive courses sensors;

FIGS. 32A-32B show images of knee braces according to various example embodiments;

FIG. 32C shows a knee brace with electrical circuit for wireless transmission according to various example embodiments;

FIG. 33A shows a soft knitted knee brace and the distribution of markers for Vicon motion capture system according to various example embodiments;

FIG. 33B shows the comparison between knee angle measured by the Vicon system versus the soft knitted knee brace according to various example embodiments;

FIGS. 34A-34D show exploded view of a sensor mounting and connections, assembled sensor, experimental setup, and block diagram of the experimental setup, respectively, according to various example embodiments;

FIG. 35A shows a graph illustrating the comparison of the sensitivity and working range of the soft knitted sensors to literature, while FIG. 35B shows a table illustrating the sensitivity and working range of the literature;

FIG. 36 shows a knitted sensor with close-up showing yarns and stitch patterns, according to various example embodiments;

FIG. 37 shows an experimental setup for sensor characterization and an assembled strain sensor according to various example embodiments;

FIG. 38A shows a response of the sensor from third cycle onwards, while FIG. 38B shows the response of the sensor when subjected to cyclic loading according to various example embodiments;

FIG. 39 shows a normalized voltage-strain relationship graph according to various example embodiments;

FIGS. 40A-40C show a knitted knee brace and reflective markers, electrical circuit schematic diagram and pulley model schematic diagram, respectively, according to various example embodiments;

FIG. 41 shows angle comparison between Vicon and middle knitted knee sensor for flexion-extension, walking and jogging according to various example embodiments;

FIG. 42 shows another schematic of stitch types according to various example embodiments;

FIG. 43 shows schematic of single needle bed stitch patterns and double needle bed stitch patterns according to various example embodiments;

FIG. 44 shows another image illustrating a knitted conductive sample mounted on linear cyclic strain test setup and close-up diagrams of the yarn according to various example embodiments;

FIG. 45 shows a schematic of another knee brace, showing the distribution of the sensor and interconnects in the wearable according to various example embodiments; and

FIG. 46 shows an image of another knee brace, showing the distribution of the sensor and interconnects in the wearable according to various example embodiments.

### DETAILED DESCRIPTION

Various embodiments of the present invention provide a textile configured for strain sensing, a method of manufacturing a textile for strain sensing and a knitting apparatus for manufacturing a textile for strain sensing.

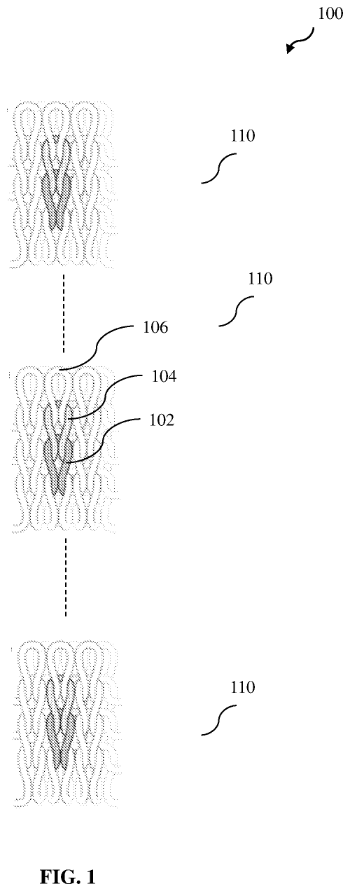

FIG. 1 depicts a schematic drawing of a textile 100 configured for strain sensing according to various embodiments of the present invention. The textile 100 comprises a strain sensing portion integrally knitted in the textile, wherein the strain sensing portion comprises a plurality of first groups 110 of courses of yarn. In particular, each first group 110 of courses of yarn comprises: a first course 102 of conductive yarn comprising a plurality of knitted loops; a second course 104 of dielectric yarn (e.g., non-conductive yarn) comprising a plurality of knitted loops; and a third course 106 of conductive yarn comprising a plurality of knitted loops. The second course 104 is arranged in between the first and third courses 102 and 106 and configured to have a higher elasticity than the first and third courses such that: when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

Accordingly, the textile 100 configured for strain sensing may be configured to include the dielectric yarn that advantageously enables contact points formation and breaking in the conductive yarn depending on the state (e.g., in a relaxed state or a stretched state) of the textile (in particular, the strain sensing portion), such as during stretching of the textile. This advantage or technical effect, or other advantages or technical effects, will become more apparent to a person skilled in the art as the textile 100 is described in more details according to various embodiments and example embodiments of the present invention.

In various embodiments, the second course is arranged in between the first and third courses and configured to have said higher elasticity than the first and third courses further such that: when the strain sensing portion is at the relaxed state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be in contact with each other, and when the strain sensing portion is at the stretched state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be not in contact with each other.

In various embodiments, the second course is arranged in between the first and third courses and configured to have said higher elasticity than the first and third courses further such that: when the strain sensing portion is at the relaxed state, for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of the second course to be in contact with each other, and for each pair of consecutive knitted loops of the plurality of knitted loops of the first course, the pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be in contact with each other, and when the strain sensing portion is at the stretched state, for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of the second course to be not in contact with each other, and for each pair of consecutive knitted loops of the plurality of knitted loops of the first course, the pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be not in contact with each other.

In various embodiments, the knitted loop of the plurality of knitted loops of the first course, the corresponding knitted loop of the plurality of knitted loops of the third course, and the corresponding knitted loop of the plurality of knitted loops of the second course are along a same wale of the strain sensing portion, and the pair of consecutive knitted loops of the plurality of knitted loops of the first course, the corresponding pair of consecutive knitted loops of the plurality of knitted loops of the third course and the corresponding pair of consecutive knitted loops of the plurality of knitted loops of the second course are along a same pair of wales of the strain sensing portion.

In various embodiments, the ratio of Young's modulus of each of the first and third courses to Young's modulus of the second course is 10³ or greater.

In various embodiments, when the strain sensing portion is at the stretched state, the strain sensing portion is stretched in a wale-wise direction (e.g., at least a component of the stretching is in the wale-wise direction).

In various embodiments, the first, second and third courses are consecutive courses.

In various embodiments, each of the first, second and third courses is formed according to a front knit-back knit stitching pattern.

In various embodiments, each intermediate first group of the plurality of first groups of courses of yarn overlaps with an immediately subsequent first group of courses of yarn and an immediately preceding first group of courses of yarn, wherein the first course of the intermediate first group is the third course of the immediately subsequent first group and the third course of yarn of the intermediate first group is the first course of the immediately preceding first group.

In various embodiments, the textile 100 may further comprise a resistor portion integrally knitted in the textile, wherein the resistor portion comprises a plurality of second groups of courses of yarn. In particular, each second group of courses of yarn comprises a first course of conductive yarn comprising a plurality of knitted loops; a plurality of second courses of dielectric yarn, each second course comprising a plurality of knitted loops; and a third course of conductive yarn comprising a plurality of knitted loops, wherein knitted loops of each pair of consecutive knitted loops of the plurality of knitted loops of the first course of conductive yarn are separated therebetween by one or more wales of dielectric yarn, knitted loops of each pair of consecutive knitted loops of the plurality of knitted loops of the third course of conductive yarn are separated therebetween by one or more wales of dielectric yarn. The plurality of second courses are arranged in between the first and third courses and said each second course is configured to have a higher elasticity than the first and third courses such that: when the resistor portion is at a relaxed state, a knitted loop of the plurality of knitted loops of the first course and a corresponding knitted loop of the plurality of knitted loops of the third course are caused by a corresponding knitted loop of the plurality of knitted loops of said each second course to be not in contact with each other, and when the resistor portion is at a stretched state, a knitted loop of the plurality of knitted loops of the first course and a corresponding knitted loop of the plurality of knitted loops of the third course are caused by a corresponding knitted loop of the plurality of knitted loops of said each second course to be not in contact with each other.

Accordingly, the textile may be configured to include the dielectric yarn that advantageously prevents the formation of contact points in the conductive yarn during stretching of the textile.

In various embodiments, the plurality of second courses are arranged in between the first and third courses and said each second course is configured to have a higher elasticity than the first and third courses such that: when the resistor portion is at the relaxed state, for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of said each second course to be not in contact with each other, and when the resistor portion is at the stretched state, for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of said each second course to be not in contact with each other.

In various embodiments, the knitted loop of the plurality of knitted loops of the first course, the corresponding knitted loop of the plurality of knitted loops of the third course, and the corresponding knitted loop of the plurality of knitted loops of said each second course are along a same wale of the resistor portion.

In various embodiments, the ratio of Young's modulus of each of the first and third courses to Young's modulus of each of the plurality of second courses is 10³ or greater. That is, for each of the plurality of second courses, the ratio of Young's modulus of each of the first and third courses to Young's modulus of the second course is 10³ or greater.

In various embodiments, when the resistor portion is at the stretched state, the resistor portion is stretched in a wale-wise direction (e.g., at least a component of the stretching is in the wale-wise direction).

In various embodiments, the first course, the plurality of second courses and the third course are consecutive courses.

In various embodiments, each of the first and third courses is formed according to a knit-miss-knit-miss with transfer stitching pattern.

In various embodiments, each intermediate second group of the plurality of second groups of courses of yarn overlaps with an immediately subsequent second group of courses of yarn and an immediately preceding second group of courses of yarn, wherein the first course of the intermediate second group is the third course of the immediately subsequent second group and the third course of yarn of the intermediate second group is the first course of the immediately preceding second group.

In various embodiments, the textile 100 further comprises a first interconnect portion integrally knitted in the textile, wherein the first interconnect portion comprises a plurality of consecutive courses of conductive yarn, each course of conductive yarn comprising a plurality of knitted loops formed according to a knit-miss stitching pattern.

In various embodiments, the textile 100 further comprises a second interconnect portion integrally knitted in the textile, wherein the second interconnect portion comprises a plurality of consecutive courses of conductive yarn, each course of conductive yarn comprising a plurality of knitted loops formed according to a knit-miss with transfer stitching pattern.

Accordingly, the textile may be configured to include the dielectric yarn that advantageously preserve the original contact points in the conductive yarn during stretching of the textile.

In various embodiments, the strain sensing portion and the resistor portion are communicatively coupled based on at least one of the first and second interconnect portions to form a circuit for measuring strain on the strain sensing portion based on changes in resistance of the strain sensing portion detected in response to the strain on the strain sensing portion.

In various embodiments, there is provided a knitted wearable comprising the textile according to any of the above-mentioned embodiments, and configured to be worn by a subject for motion sensing of the subject (e.g., a knee brace worn at a knee portion of the subject for motion sensing related to the knee of the subject).

Accordingly, various embodiments provide a highly stretchable electrically conductive textile through the mechanical coupling of an electrically conductive and a dielectric yarn in a stitch pattern in which the dielectric yarn functions to control the electrical contact points in the conductive yarn in a configured one of the following manner: the dielectric yarn enables periodic contact points formation and breaking in the conductive yarn during stretching of the textile (e.g., when configured as a strain sensing portion), the dielectric yarn preserve the original contact points in the conductive yarn during stretching of the textile (e.g., when configured as a resistor portion), and/or the dielectric yarn prevent the formation of contact points in the conductive yarn during stretching of the textile (e.g., when configured as an interconnect portion).

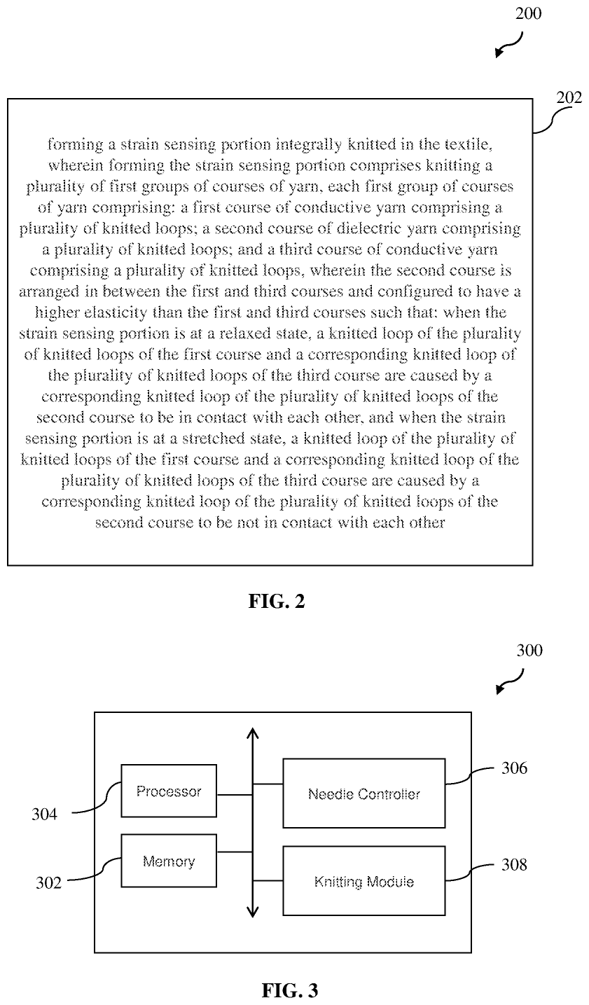

FIG. 2 depicts a schematic flow diagram of a method 200 of manufacturing a textile for strain sensing. The method 200 comprises: forming (at 202) a strain sensing portion integrally knitted in the textile, wherein forming the strain sensing portion comprises knitting a plurality of first groups of courses of yarn, each first group of courses of yarn comprising: a first course of conductive yarn comprising a plurality of knitted loops; a second course of dielectric yarn comprising a plurality of knitted loops; and a third course of conductive yarn comprising a plurality of knitted loops, wherein the second course is arranged in between the first and third courses and configured to have a higher elasticity than the first and third courses such that: when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

In various embodiments, the method 200 is for manufacturing the textile for strain sensing 100 as described hereinbefore with reference to FIG. 1, therefore, the method 200 may further include various steps corresponding to providing or forming various configurations and/or components/portions of the textile 100 configured for strain sensing as described herein according to various embodiments, and thus such corresponding steps need not be repeated with respect to the method 100 for clarity and conciseness. In other words, various embodiments described herein in context of the textile for strain sensing 100 is analogously or correspondingly valid for the method 200 (e.g., for manufacturing the textile for strain sensing 100 having various configurations and/or components/portions as described herein according to various embodiments), and vice versa.

FIG. 3 depicts a schematic block diagram of a knitting apparatus 300 (e.g., Computer Numerical Control (CNC) knitting machine) for manufacturing a textile for strain sensing according to various embodiments, such as the textile configured for strain sensing 100 as described hereinbefore with reference to FIG. 1 according to various embodiments. The system 300 comprises a memory 302, at least one processor 304, and a needle controller 306 configured to control one or more needles for performing knitting (or stitching). The at least one processor 304 is communicatively coupled to the memory 302 and the needle controller 306, and configured to perform a method of manufacturing a textile configured for strain sensing 100 as described hereinbefore with reference to FIG. 1 according to various embodiments.

It will be appreciated by a person skilled in the art that the processor 304 may be configured to perform the required functions or operations through set(s) of instructions (e.g., software module(s)) executable by the processor 304 to perform the required functions or operations. Accordingly, as shown in FIG. 3, the knitting apparatus 300 may comprise a knitting module (or a knitting circuit) 308 configured to control the needle controller 306 to perform a method of manufacture a textile configured for strain sensing 100 as described hereinbefore with reference to FIG. 1 according to various embodiments.

It will be appreciated by a person skilled in the art that the above-mentioned module may be realized by or implemented as one functional module (e.g., a circuit or a software program) as desired or as appropriate. For example, the knitting module 308 may be realized as an executable software program (e.g., software application or simply referred to as an “app”), which for example may be stored in the memory 302 and executable by the processor 304 to perform the functions/operations as described herein according to various embodiments.

For example, in various embodiments, the memory 302 may have stored therein the knitting module 308 as described hereinbefore according to various embodiments, which are executable by the processor 304 to perform the corresponding functions/operations as described herein.

A computing system, a controller (e.g., microcontroller) or any other system providing a processing capability may be provided according to various embodiments in the present disclosure. For example, the knitting apparatus 300 described hereinbefore may include a processor (or controller) 304 and a computer-readable storage medium (or memory) 302 which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions which will be described in more detail below may also be understood as a “circuit” in accordance with various alternative embodiments. Similarly, a “module” may be a portion of a system according to various embodiments in the present invention and may encompass a “circuit” as above, or may be understood to be any kind of a logic-implementing entity therefrom.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

In addition, the present specification also at least implicitly discloses a computer program or software/functional module, in that it would be apparent to the person skilled in the art that the individual steps of the methods described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention. For example, it will be appreciated by a person skilled in the art that the knitting module 308 may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, one or more of the steps of a computer program/module or method described herein may be performed in parallel rather than sequentially. Such a computer program may be stored on any computer readable medium. The computer readable medium may include storage devices such as magnetic or optical disks, memory chips, or other storage devices suitable for interfacing with a computer processor. The computer program when loaded and executed on such a computer processor effectively results in an apparatus or device that implements one or more steps of the methods described herein.

The software or functional modules described herein may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the software or functional module(s) described herein can also be implemented as a combination of hardware and software modules.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Any reference to an element or a feature herein using a designation such as “first”, “second” and so forth does not necessary limit the quantity or order of such elements or features, unless stated or the context requires otherwise. For example, such designations may be used herein as a convenient way of distinguishing between two or more elements or instances of an element. Thus, unless stated or the context requires otherwise, a reference to first and second elements does not necessarily mean that only two elements can be employed, or that the first element must precede the second element. In addition, a phrase referring to “at least one of” a list of items refers to any single item therein or any combination of two or more items therein.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

Various example embodiments provide seamlessly integrated stretchable circuits in all knitted soft wearables.

Textile based soft wearables may have high stretchability, breathability and soft hand feel, and provide a platform for the development and integration of sensing functions into a soft wearable. The components of a soft wearable may include (a) sensors and (b) a network of stretchable electrical components that carry signals to-and-fro. Most of the existing works that have demonstrated both sensing capabilities and an electrical circuit in a textile wearable have achieved it through external integration of conductive fabric or conductive yarn using sewing or embroidery techniques. As a result, the electrical and mechanical properties of the sensors and other electrical components are often limited by the overlying conductive fabric or the conductive yarn. According to various embodiments of the present invention, knitted stretchable conductive knits are provided, the macroscopic electrical and mechanical properties of which can be tuned by varying the properties of the yarn at the micro scale and the arrangement of the stitch types in the unit cell at the meso scale. Through suitable arrangement of the yarn and stitch types in the unit cell, electrical components, such as interconnects and resistors, were knitted that demonstrate negligible change in electrical resistance in response to strain. The interconnects demonstrate a very low initial resistance of about 0.4 ohm with negligible change in voltage for strains up to 140%, while the resistor demonstrates a small change of about 2.5 ohm from its initial resistance of 25.71 ohm when stretched up to 250% strain. A stretchable strain sensor of high sensitivity (e.g., gauge factor of about 8.96) and working range (e.g., about 68%) was also developed according to various embodiments. In various example embodiments, the strain sensors, resistors and interconnects were distributed and knitted as a single holistic piece in a soft wearable knee brace for joint motion sensing. The development of a network of knitted stretchable electrical components, such as resistors and interconnects, and their integration with other sensing components forming a single piece of knitted conductive fabric has not been demonstrated before.

According to various example demonstrations, all knitted stretchable circuits for all kinds of soft wearables may be provided. Some examples include soft wearables for motion sensing (e.g., human joints or other moving parts), monitoring of physiological signals (e.g., respiration and heart rate), prognosis of healthcare conditions, disease trajectory tracking, rehabilitation, posture tracking, sensing of speech disorders, gesture recognition, etc.

Various example embodiments provide wearable devices can be designed as a single knit from scratch by varying the geometric and material properties of the conductive fabric, using Computer Numerical Control (CNC) knitting technology. The CNC knitting machine is analogous to a multi-material 3D printer as it can seamlessly integrate multiple yarns in various stitch patterns and stitch densities into a single textile of arbitrary 3D geometry. This produces a bespoke textile in a single manufacturing step which minimizes the need for post-processing methods, such as cutting or sewing. Most previous works on knitted conductive textiles have focused on understanding the physics behind the electromechanical response of the conductive knit by either developing electromechanical models to predict their resistance or investigating the effect of knitting parameters on the sensitivity of the conductive knit. Some works have proposed methods to optimize the contact resistance in electrically conducting textiles for sensing applications. However, the development of stretchable electrical components such as interconnects and resistors and their integration into an all knitted soft wearable has not been demonstrated before. There has been limited work on the development of embedded soft knitted wearables in a single holistic knit.

In various example embodiments, highly stretchable knitted electrical components of (a) sensors and (b) a network of interconnects and resistors and their integration into an all knitted soft wearable, for example, a knee brace for joint motion sensing, is provided. The desirable properties for a sensor, and an interconnect or a resistor are greatly contrasting in that it is preferable for a strain sensor to produce a large change in resistance for enhanced sensitivity. On the other hand, an interconnect or a resistor should produce negligible change in resistance to the transmitted signal when stretched. In addition, the resistor may have a higher resistance while an interconnect may have as small a resistance as possible. These vastly different electro-mechanical responses may be obtained within the same conductive fabric by locally varying the macroscopic electrical property of the conductive knit, through combined variations of the underlying yarn and the stitch pattern.

### Principle of Resistance Change in Conductive Knits

According to various example embodiments, the conductive yarn (e.g., nylon yarn) employed shows negligible stretchability by itself and therefore demonstrates a limited change in resistance when the yarn alone is stretched. However, the conductive knit formed from this yarn may be designed for high stretchability with desired resistance change by tuning the geometry of the looped structure and the contact resistance. As the knit is stretched, the loops pull against each other which elongates the loops and reconfigures their contact points. According to the Holm's contact theory as described in Holm, R., Electric contacts: theory and application. 2013: Springer Science & Business Media, contact resistance between two conductive electrodes is given by

$${R}_{c} = \frac{ρ}{2} \sqrt{\frac{π H}{n P}} ,$$

where ρ is the resistivity of the material, H is the material hardness, n is the number of contact points and P is the contact pressure. Hardness and resistivity are material properties that remain unchanged. However, upon stretching, the contact resistance varies with the change in the number of contact points and contact pressure.

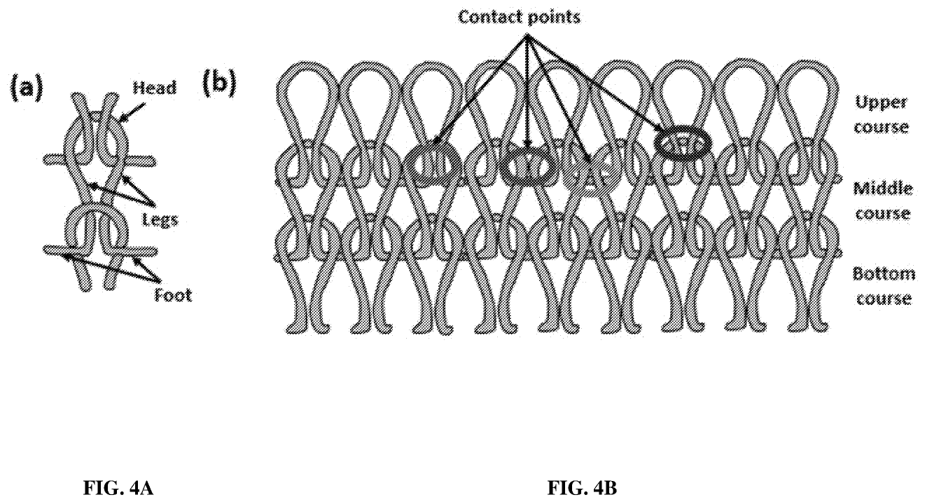

FIG. 4A shows a schematic of a knitted loop according to various example embodiments. More particularly, FIG. 4A shows the structure of a single knitted loop in a conductive knit of a simple purl stitch pattern. FIG. 4B shows the arrangement of several knitted loops at rest state. A single row of the conductive fabric is called a course and a single column is called a wale. More particularly, FIG. 4B shows various contact points in a stitch pattern. As the loops are knitted with interlocked structures, there are multiple contact points where the courses (rows) and wales (columns) touch each other. It is understood that the macroscopic electrical property of the knitted conductive fabric depends upon properties of the yarn material at the micro scale and stitch pattern of the knitted loops at meso scale. For a given conductive yarn, the electrical property of the knitted fabric may depend upon three factors: 1) geometry of the knitted loops (or stitch pattern), 2) stitch density, and 3) contact resistance. The knitted loops can be arranged in a variety of stitch patterns as described in Tan, Ying Yi, “Graded Knit Skins: Design & Fabrication of Modular Membrane Façade Panels Using Bespoke Multi-Material Knitted Textiles Shaped by Bendable Rod Assemblies”, 2020, PhD Thesis, Singapore University of Technology and Design: Singapore (https://sutd.primo.exlibrisgroup.com/permalink/65SUTD_INST/19hmrhl/alma99958 3164602406), and further altered by controlling the stitch density of the loops. This provides a unique capability to control the electrical property of knitted fabrics by virtue of the underlying yarn material, stitch patterns and stitch density, thus providing a way to optimize their physical properties for desired functionality.

Because of this flexibility in the design of conductive knitted fabrics, a single knitted piece can possess vastly varying electrical and mechanical properties. For example, various example embodiments demonstrate that by using a combination of suitable yarn and stitch patterns, the conductive fabric can be designed as a strain sensor, a resistor or an interconnect. Various example embodiments further demonstrate that these three components can be distributed within a single piece of the knitted soft wearable in a single knit job.

### Stretchable Circuits: Sensors, Resistors and Interconnects

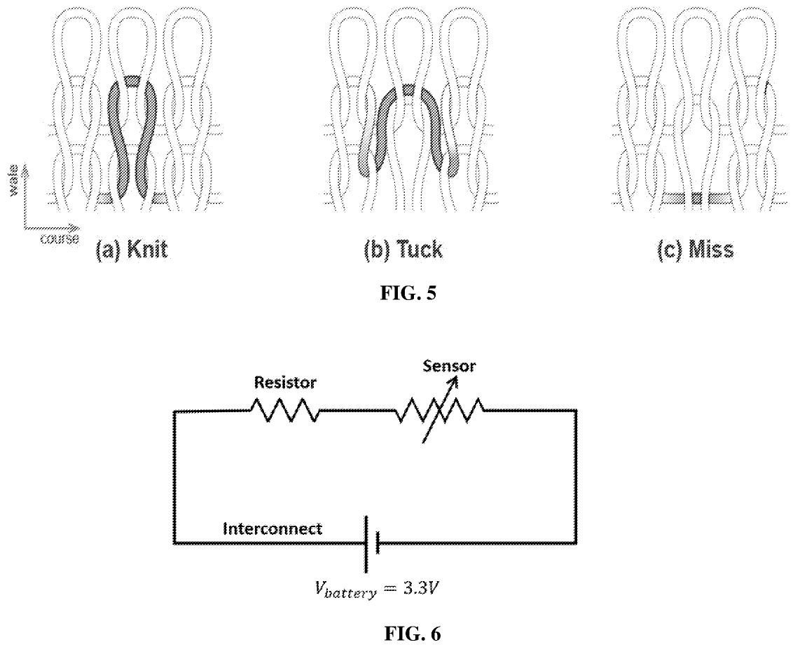

As discussed above, the Holm's theory gives the relationship between the contact resistance and the number of contact points and contact pressure change. Therefore, for a stitch pattern, a greater number of contact points results in a lower contact resistance and vice-versa. FIG. 5 shows basic stitch types forming a conductive loop. More particularly, FIG. 5 shows three basic stitch types, such as knit stitch, tuck stitch and miss stitch, that are combined to form stitch patterns. The stitch patterns are a unit cell of knitting instructions that repeat periodically to form a knitted fabric. Since these basic stitch types form the basis of the stitch pattern, their arrangement plays an important role in determining the resulting electrical resistance of the conductive knit. For a conductive knit with stitch types shown in FIG. 5, both the knit and tuck stitches result in a substantial number of contact points between adjacent loops, while a miss stitch exhibits the least number of contact points. Therefore, in general, increasing the number of knit and tuck stitches in an all-conductive knit decreases the overall resistance of the conductive fabric. However, generalization cannot be made for the miss stitch since the contact points also depends upon the arrangement of other stitch types with respect to the miss stitch in the unit cell, as described in Liu, Su, “A study of the influence of different structures on resistivity of conductive knitted fabrics”. 2017, PhD thesis, Hong Kong Polytechnic University (https://theses.lib.polyu.edu.hk/handle/200/8928). Contrastingly, to increase the overall resistance conductive loops can be separated with non-conductive loops or alternatively, adjacent conductive loops can be separated with a miss stitch, resulting in a lower number of contact points between conductive loops. Accordingly, from these examples, the freedom to distribute the different stitch types along with a suitable combination of yarns (both conductive and non-conductive (or dielectric)) in the design of the unit cell provides a distinctive way to control the distribution of contact points in the conductive knit and hence control its overall resistance. According to various example embodiments, three different stitch patterns used as interconnects, resistors and sensors, respectively, within the same conductive knit is described. The resistors, sensors and interconnects are then distributed and knitted as a single holistic piece of knit according to the circuit diagram shown in FIG. 6, forming a soft wearable knee brace for joint motion sensing, in a non-limiting example. More particularly, FIG. 6 shows a circuit diagram of the soft wearable knee brace showing resistor and sensor connected via interconnects, according to various example embodiments.

### Strain Sensors

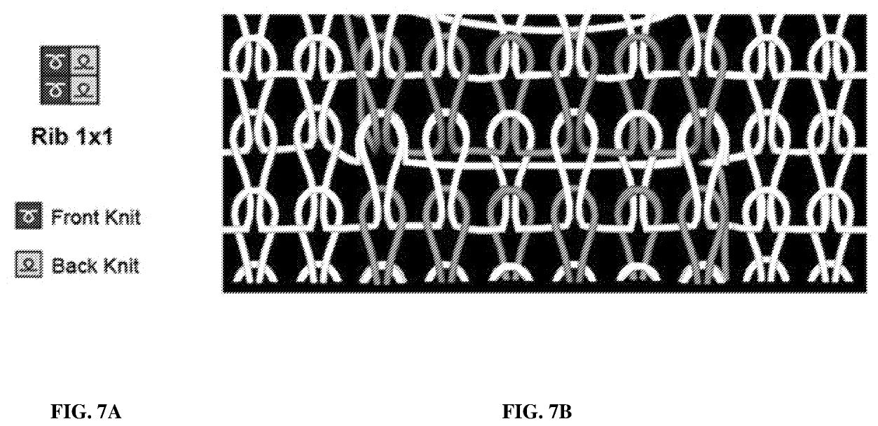

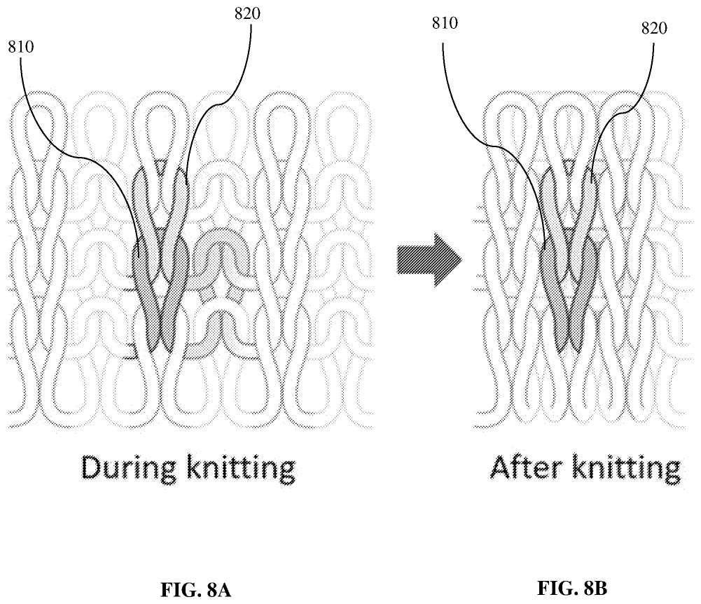

For strain sensors, it is desirable for the sensor to demonstrate a significant change in resistance when stretched. The contact resistance at contact points of the conductive knit has a major influence on its overall resistance. Hence, through a targeted distribution of the yarns and stitch types within the unit cell of the conductive knit, the number of contact points and their behaviour in response to stretch may be tuned. Thus, the sensitivity of the sensor may be tuned. FIG. 7A shows a unit cell of the knit instruction according to various example embodiments. In terms of the stitch types, the alternating wales of front and back knits are arranged within the unit cell. FIG. 7B shows a graphical simulation of the knitted loops for a unit cell containing alternate courses of conductive and non-conductive yarn. More particularly, FIG. 7B shows a graphical simulation of the wale-wise strain sensor stitch pattern. The grey lines represent the conductive yarn while the white lines represent the non-conductive yarn (corresponding to the dielectric yarn as described hereinbefore according to various embodiments). Compared to a knit with wales of only front knits or only back knits, the alternate arrangement of front and back knits (corresponding to the “front knit-back knit stitching pattern” as described hereinbefore according to various embodiments) allows the knitted conductive fabric to fold upon itself in the width direction, resulting in a partial overlap between the front and back knits at the rest state. FIGS. 8A-8B show the schematic of the front (prominent) and back knits (faded) during the knitting process and at the rest state, respectively. In terms of the yarns, different distributions of the conductive and non-conductive yarn within the unit cell may be targeted. More particularly, FIGS. 8A-8B show the schematic of the knitted loops in the conductive knit during knitting and at rest state after knitting, respectively. The yarn 810 represents the conductive yarn, and the yarn 820 represents the non-conductive yarn.

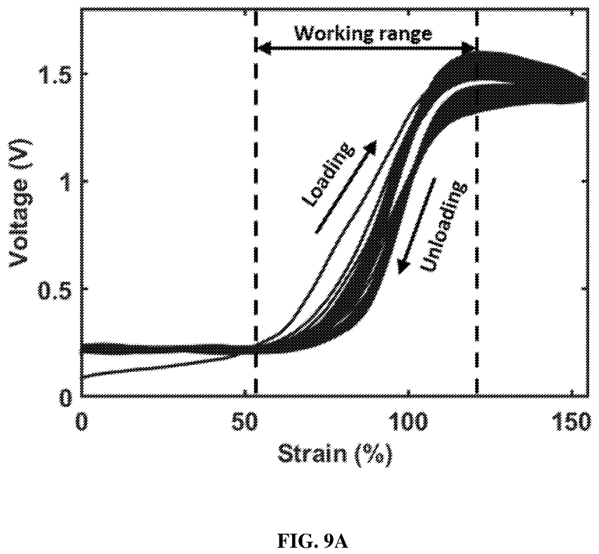

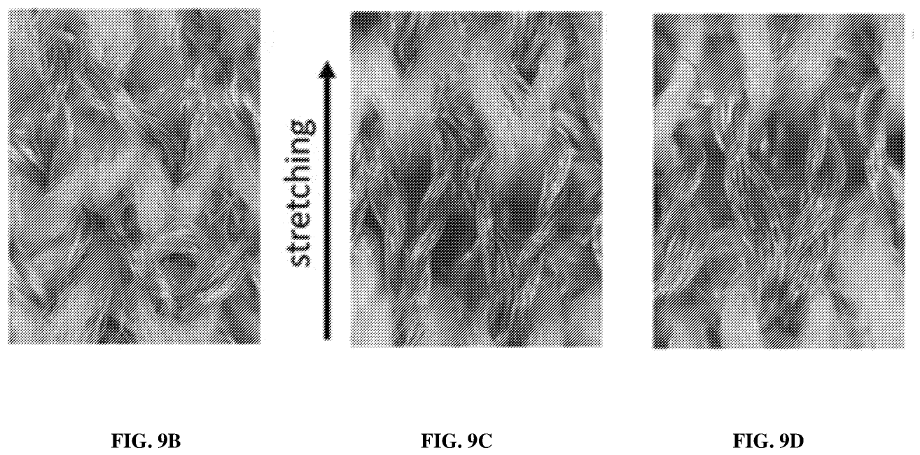

FIG. 9A shows a graph of voltage vs strain response of the alternate conductive and non-conductive knit. More particularly, FIG. 9A shows the change in voltage of the strain sensor for the combination with alternate conductive and non-conductive courses, when it is stretched. As can be seen, the voltage first increases (working range), followed by a plateau after a certain strain value. This is because initially the conductive loops move away from each other in both the course and wale directions, resulting in a drop in contact pressure and eventually breaking off the contact points. Further stretching of the sensor results in the loops getting longer and narrower without creating new contact points.

FIGS. 9B-9D show microscopic images of the alternate conductive and non-conductive knit at rest state, 100% strain and 125% strain, respectively. FIG. 9B shows the arrangement of the front face of the loops at the rest state. As can be seen, the front wales are collapsed together, with heads of the conductive knitted loops in close contact. Similarly, the foot and head of the nearest conductive courses touch each other. As the sensor is stretched, the nearest courses start to move away from each other and so do the front wales (and back wales). FIG. 9C shows the courses moving away from each other when stretched to 100% strain. Further stretching to 125% strain leads to the wale contact points breaking off, as shown in FIG. 9D. As the number of contact points and contact pressure changes with increasing strain, it results in an increase in the contact resistance and hence the overall resistance of the fabric.

In various example embodiments, the super elastic dielectric yarn alternates between adjacent courses of the conductive yarn in the unit cell. At the relaxed state, the nearest conductive wales and courses are in close contact owing to the high elastic forces from the dielectric yarn. As the fabric is stretched, the loops of the super elastic dielectric yarn stretch significantly while also becoming longer and narrower. The conductive yarn loops become longer and narrower with little stretch owing to their low elasticity, thus breaking the wale wise and course wise contacts (FIGS. 9C and 9D). This cycle repeats as the fabric is stretched cyclically.

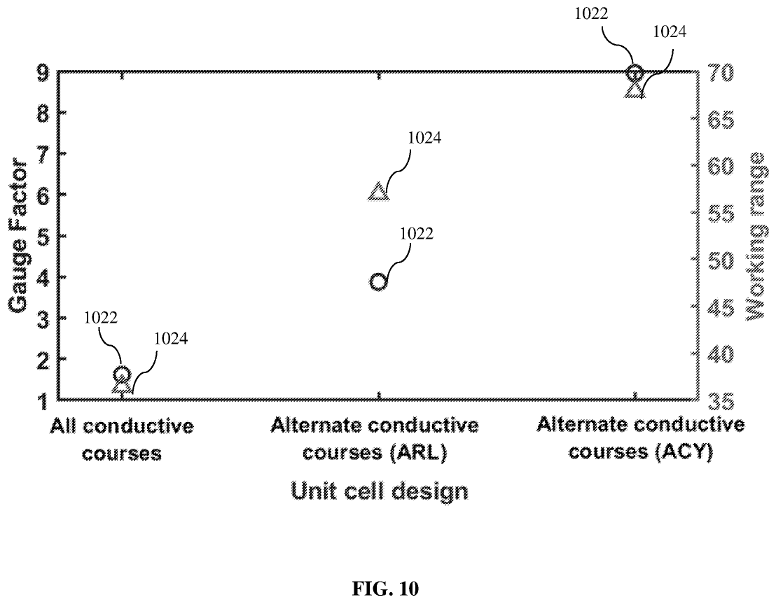

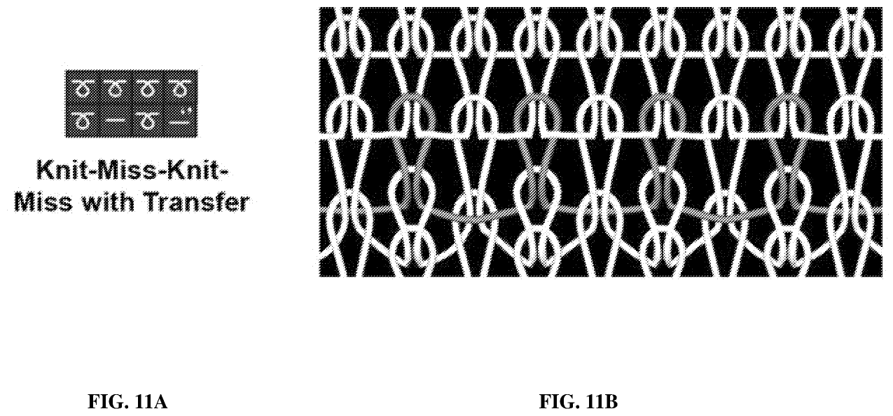

FIG. 10 shows the comparison of the sensor's performance among conductive knits with different yarn material combinations. More particularly, FIG. 10 shows a graph illustrating a comparison of performance between the unit cell with all conductive and alternate conductive course sensors. In the first case, the unit cell consists of an all-conductive course. The second and third knits consists of a unit cell with alternate courses of conductive and non-conductive yarn, however in the second knit a less elastic acrylic yarn (ARL) is used as the non-conductive yarn while in the third knit a highly stretchable air covered spandex (ACY) yarn is used to separate the conductive courses. As can be seen from FIG. 10, the gauge factor 1022 and working range 1024 of the alternate conductive course sensor is significantly higher than all conductive course sensor. This is because in an all-conductive course knit, the conductive loops are tightly interlocked with each other with fewer contact points that can potentially break off when stretched. In contrast, the unit cell with alternate courses of non-conductive yarn has a greater number of contact points that can potentially break off (e.g., no longer forming contact points) when stretched (e.g., corresponding to the “a knitted loop of the plurality of knitted loops of the first course and a corresponding knitted loop of the plurality of knitted loops of the third course are caused by a corresponding knitted loop of the plurality of knitted loops of the second course to be not in contact with each other” as described hereinbefore according to various embodiments). This results in a larger change in resistance and higher sensitivity, as evident from the gauge factor which increases by as much as 2.4× when the conductive courses are separated by a non-conductive acrylic yarn. A better separation of the contact points upon stretching is obtained by using a highly stretchable air covered spandex yarn (ACY) which results in a 5.52× increase in sensitivity of the sensor and 1.86× increase in working range, compared to all conductive courses. This is because, a highly elastic yarn such as ACY ensures good contact between the nearest conductive courses and wales at the rest state due to its elastic pull (e.g., corresponding to the “when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other” and “when the strain sensing portion is at the relaxed state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be in contact with each other” as described hereinbefore according to various embodiments). However, when stretched, it simultaneously allows for a better separation between the conductive courses owing to its high stretchability (e.g., corresponding to the “when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other” and “when the strain sensing portion is at the stretched state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be not in contact with each other” as described hereinbefore according to various embodiments).

### Resistors

According to various example embodiments, a simple potential divider circuit with a fixed resistor and a sensor may be used to read the voltage difference across the sensor, as shown in FIG. 6. For the circuit to perform reliably, it is desirable for the resistor to maintain a known fixed resistance under stretch. It is noted that the resistivity of the conductive yarn poses an upper limit to the overall resistivity of the conductive knit, as any contact between the yarn will result in lowering of its overall resistance. Also, designing the resistor as a straight line, for example as an inlay, would reduce the length of the resistor and compromise its stretchability. To achieve both stretchability and consistent performance under stretch, the conductive knit stitches may be staggered within the unit cell of the conductive fabric. The staggering of tuck stitches was avoided due to reliability issues while knitting large number of tuck stitches. FIG. 11A shows a unit cell of knit instruction. More particularly, FIG. 11A shows the unit cell which has conductive knit stitches separated by miss stitches. As shown, FIG. 11A includes knit-miss-knit-miss with transfer stitching pattern. FIG. 11B shows a graphical simulation of the stitch pattern in conductive knit. The grey lines represent the conductive yarn while the white lines represent the non-conductive yarn. More particularly, FIG. 11B shows the graphical simulation of the knitted loops with the unit cell. As can be seen, the conductive knit stitches (grey) are separated by the non-conductive surrounding yarn (white).

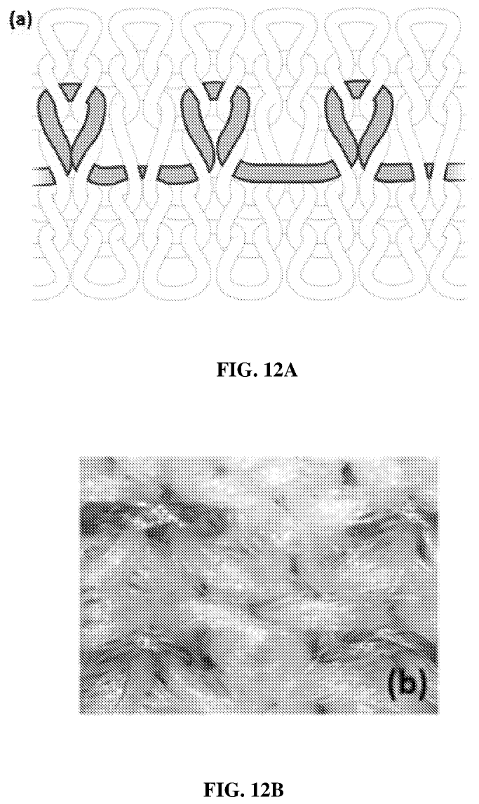

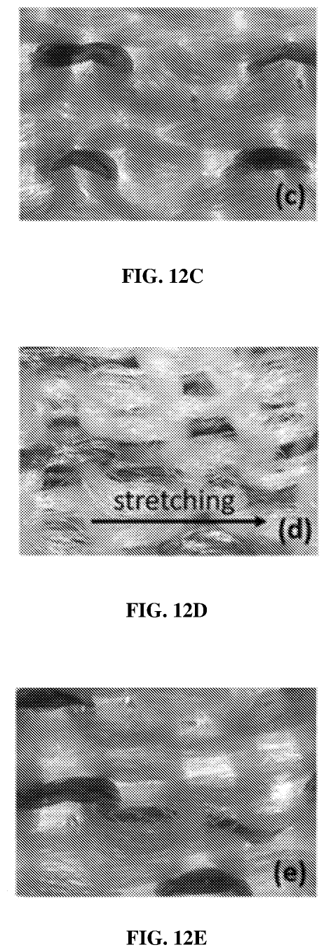

It is desirable for a resistor in the conductive fabric to maintain known fixed resistance under stretch. It's also significant to note that the resistivity of the conductive yarn poses an upper limit to the overall resistivity of the conductive knit, as any contact between the yarn will result in lowering of its overall resistance. Also, designing the resistor as a straight line, for example as an inlay reduces the length of the resistor and compromises its stretchability. To achieve both stretchability and consistent resistor performance under stretch, the conductive knit stitches are separated by the super elastic dielectric yarn in both courses as well as in wales as shown within the unit cell of the conductive fabric as illustrated in FIG. 12A.

FIG. 12A shows a schematic of the course wise resistors with staggered knit stitch. More particularly, FIG. 12A shows the schematic of the course-wise resistors with knit-miss stitch. FIGS. 12B-12E show microscope images of several courses of the resistor stitch pattern, with each resistor course insulated from the adjacent resistor courses by courses knitted with non-conductive yarn. FIG. 12B shows the course wise resistor at the rest state under ambient light. The samples were then backlit with strips of blue LED lights (backlit condition), as shown in FIG. 12C. Because of the differential absorption of the blue light, the underlying loop geometry can be seen to a certain extent. FIGS. 12D-12E show the stretched state of the resistors at 50% strain under ambient light and backlit condition, respectively. From FIG. 12C and FIG. 12E, it can be seen that the heads and legs of the loops remain well separated at both the rest and the stretched state. Moreover, the knit stitch helps to improve stretchability of the resistor as the conductive yarn tends to straighten out when stretched.

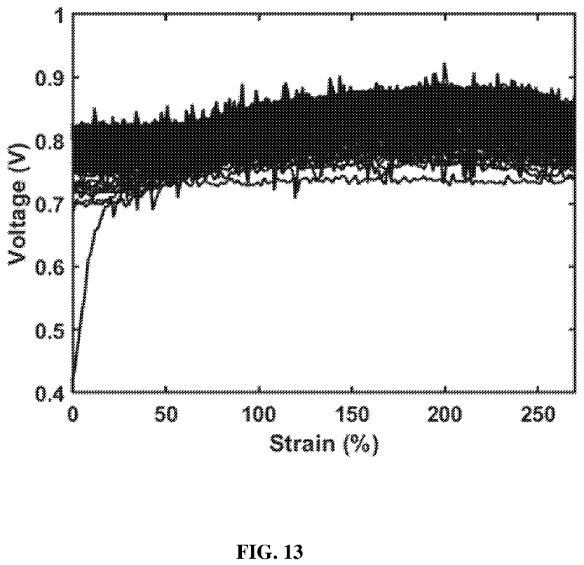

FIG. 13 shows the voltage vs strain graph of the course wise knitted resistor. The mean resistance of the resistor was found to be about 25.71 ohm. Even when the resistor is stretched to more than 250% strain, it shows a small change in resistance of about 2.5 ohm.

### Interconnects

Interconnects are the electrical components that transmit signals to-and-fro forming a network of electrical circuit. Some of the requirements for stretchable interconnects are, first low resistance to ensure minimal change to the transmitted signal, and second negligible change in resistance when stretched. To address this, a highly conductive yarn coupled with a unit cell with a greater number of contact points may be used, to reduce the overall resistance of the fabric.

Both the course wise and wale wise knitted interconnects will be described. The course wise knitted interconnect may be designed to provide horizontal connections, while the wale wise knitted interconnect may be designed to provide vertical connections.

### Course Wise Interconnect

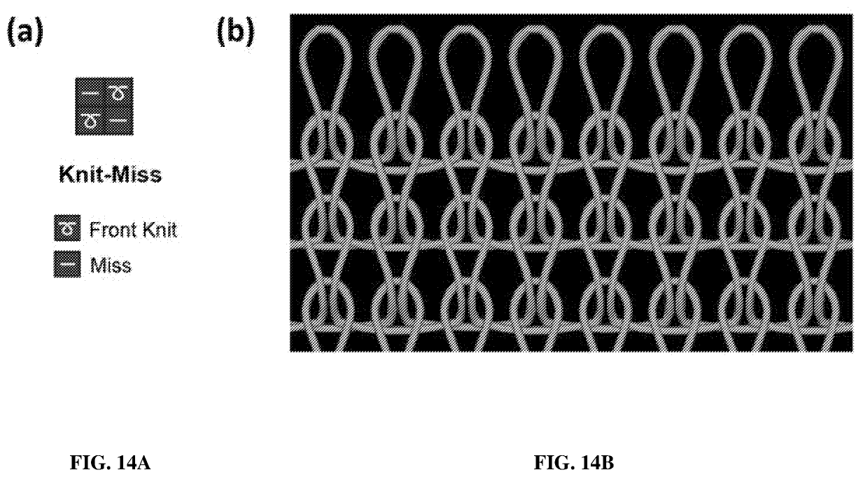

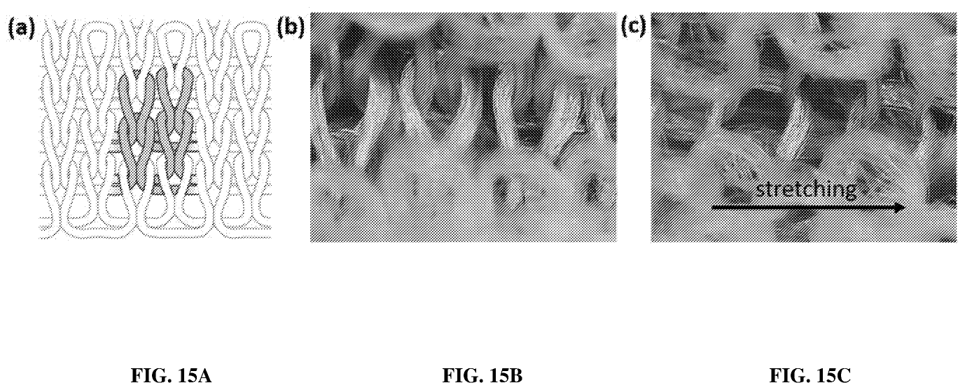

From previous results, it is noted that the interlocked loop structure results in a lower change in resistance when stretched. Moreover, a unit cell with a combination of all conductive knit and miss stitches results in an increased number of contact points compared to a unit cell with only knit stitches. According to various example embodiments, an all conductive knit and miss stitch for the course wise interconnect may be employed, as shown in FIG. 14A. More particularly, FIG. 14A shows a unit cell of knit instruction. FIG. 14A shows a knit-miss stitching pattern. FIG. 14B shows a graphical simulation of the knitted loops with the unit cell. More particularly, FIG. 14B shows a graphical simulation of the stitch pattern in conductive knit. As can be seen, all adjacent wales and courses are knitted with conductive yarn (grey). Moreover, the miss stitches at the bottom of the knitted loops provide enhanced contact between the adjacent courses and wales. FIG. 15A shows the schematic of the unit cell displaying the contact points. More particularly, FIG. 15A shows a schematic of the course wise interconnects. FIGS. 15B-15C show microscopic images of the course wise interconnects at rest state and 50% strain, respectively. More particularly, FIG. 15B shows the knitted loops in a course wise knitted interconnect at rest state. As can be seen, it exhibits minimal contact between the heads of the adjacent wales. FIG. 15C shows the stretched state of the knitted loops at 50% strain. The wales can be seen to move farther away in response to the course wise stretching.

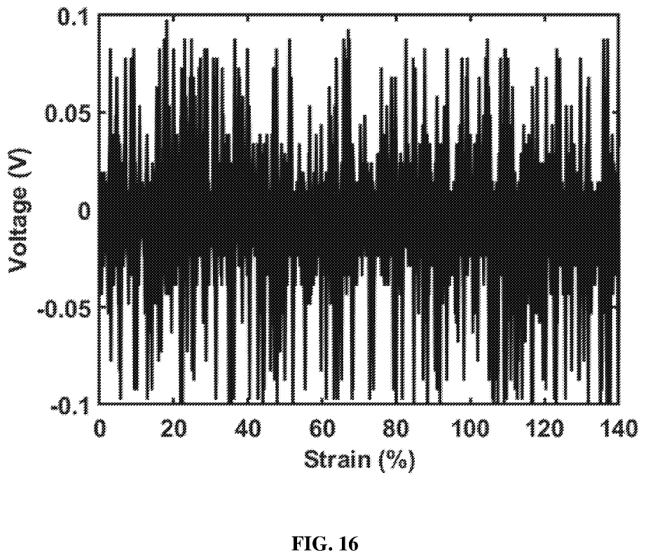

FIG. 16 shows the voltage vs strain graph for the course wise knitted interconnect. Owing to the high conductivity of the conductive yarn and minimal change in number of contact points, a very low resistance and resistance change is observed. The mean initial resistance of the interconnect was found to be about 0.4 ohm. As the interconnect is stretched up to 140%, it shows negligible change in resistance.

### Wale Wise Interconnect

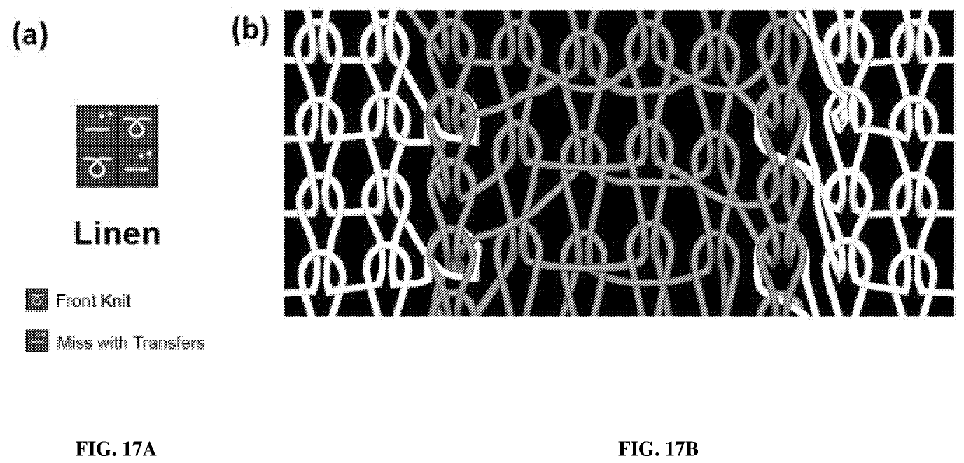

FIG. 17A shows the unit cell for the wale wise interconnect. More particularly, FIG. 17A shows a unit cell of knit instruction. The interconnect has all conductive courses of knit and miss with transfer stitches (in grey) (e.g., corresponding to the “knit-miss with transfer stitching pattern” as described hereinbefore according to various embodiments), as shown by the graphical simulation of the knitted loops in FIG. 17B. More particularly, FIG. 17B shows a graphical simulation of the stitch pattern in conductive knit. The grey lines represent the conductive yarn while the white lines represent the non-conductive yarn. The miss with transfer stitch is made by transferring the needle loop to the opposite needle bed before the miss stitch, so that the float yarn of the miss stitch appears on the technical face of the fabric. The interconnect is integrated into the surrounding fabric (in white).

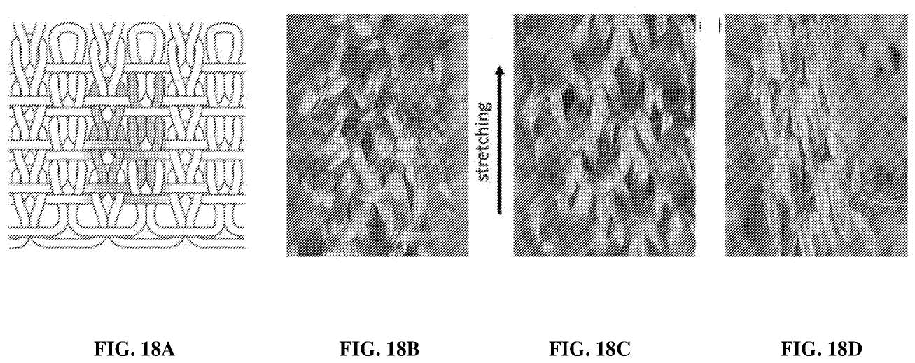

It is desirable for an interconnect that serves as the medium to transmit signal in a conductive fabric to stretch along with the fabric; however, it should be accompanied with a negligible change in resistance when stretched to ensure minimal change to the transmitted signal. A highly conductive yarn coupled with a unit cell with a greater number of contact points may be used, to reduce the overall resistance of the fabric. The interconnect is integrated into the super elastic dielectric yarn as the surrounding fabric (in white). FIG. 18A shows the schematic of the unit cell of the wale wise interconnects displaying the contact points. As can be seen, the miss stitch results in an increased number of contact points between the adjacent wales and courses. FIG. 18B shows the conductive loops in a wale wise knitted interconnect at rest state. The conductive loops are compressed together at the rest state due to high elastic forces from the super elastic dielectric yarn. FIG. 18C and FIG. 18D show the loops when the interconnect is stretched by 50% and 75% strain, respectively. As can be seen, when the interconnect is stretched in the wale wise direction, the adjacent wales move closer, ensuring a good contact.

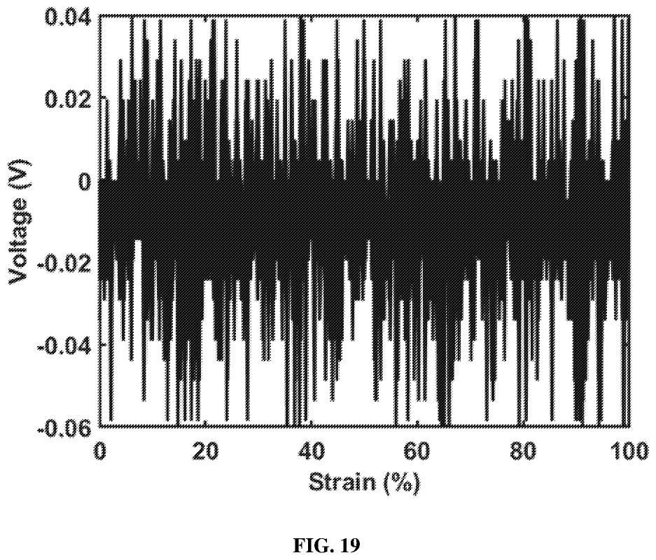

FIG. 19 shows the voltage vs strain graph for the wale wise knitted interconnect. A very low resistance and resistance change is observed. The mean initial resistance of the interconnect was found to be about 0.3 ohm. As the interconnect is stretched up to 100% strain, it shows negligible change in resistance.

### Example Applications: Soft and Stretchable Knee Brace

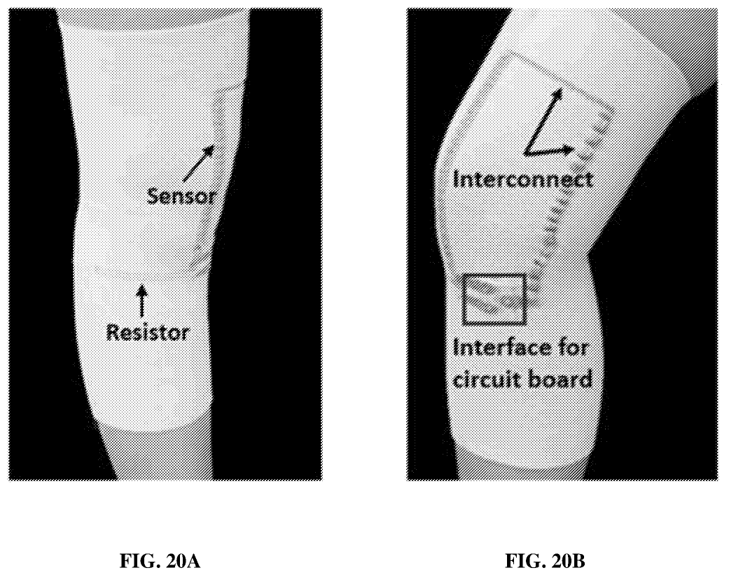

According to various example demonstrations, the above-mentioned electrical components may be programmed into a CNC knitting machine and distributed on a soft and stretchable wearable, for example, a knee brace. The knee brace is shaped as a tube and is knitted with elastic spandex yarn for stretchability so that the knee brace can adapt to the shape of the user's knee. FIGS. 20A-20B show a left side view, and a right side view of the knee brace, showing the distribution of the sensor, interconnects and resistor in the wearable. As shown in FIGS. 20A-20B, the strain sensor measures the deformation and transmits the signal to the interconnects which in turn carry the signal to the circuit board with an Analog to Digital Converter (ADC) and wireless transmitter. The knitted resistor is used as a potential divider, thus completing the circuit. With the wearable, the knee joint motion may be effectively sensed and transmitted wirelessly to a smartphone or other device.

### Materials and Knitted Structure of Knee Brace

According to various example demonstrations, most of the knee brace is knitted from a white spandex air covered yarn (e.g., 100D spandex with 300D polyester DTY), as shown in FIG. 21. More particularly, FIG. 21 shows an all knitted soft wearable knee brace with all the electrical components integrated as a single holistic device. For example, it is knitted with purl (garter) stitch pattern so that the edges lie flat and resist curling.

### Wale-Wise Strain Sensor

The wale-wise strain sensor is located along the centre of the knee so that the sensor is subject to a large strain when the user bends his/her knee. The strain sensor is knitted using rib 1×1 stitch pattern with two yarns—a conductive silver-coated polyamide yarn and the same non-conductive spandex air covered yarn that is used to knit the rest of the knee brace. This combination allows the strain sensor to have a high gauge factor and large working range so that it can precisely sense the bending of the knee joint.

### Interconnects

The interconnects are knitted with a PBO yarn with silver cladding that has a very low resistance. The course-wise interconnects are knitted with knit-miss while the wale-wise interconnects are knitted with linen stitch, because these two stitch patterns were found to have low gauge factors when stretched course-wise and wale-wise, respectively. The combination of low resistance and low gauge factors means that the interconnects have a low and constant resistance regardless of strain.

### Resistor

The resistor is knitted with the same conductive silver-coated polyamide yarn that is used to knit the strain sensor. It is knitted with a knit-miss-knit-miss with transfer stitch pattern that has minimal contact points between the conductive yarn loops to increase the resistance and reduce the change in contact resistance when the resistor is stretched. To achieve the required resistance for the circuit, multiple courses of the resistor can be knitted to increase the length and thus resistance.

### Connection to Printed Circuit Board (PCB)

According to various example demonstrations, to connect to the PCB, a pocket is seamlessly knitted into the knee brace. The knitted interconnects are knitted onto the outer surface of the pocket, and the PCB ground, power, and signal pins/patches can contact the knitted interconnects when the PCB is inserted into the pocket.

Wearables made from conductive fabric (CF) or e-textiles have received growing interest for several sensing applications, including joint motion sensing, respiratory monitoring, etc. In most of these applications, CFs have been designed into a piezoresistive sensor due to their highly elastic property, and they exhibit intrinsic resistance change when they are stretched. These CF-based sensors are light weight, non-invasive and can be integrated into the garment, providing more comfort to the wearer. These advantages enable a potential use of CF sensors for long-term continuous sensing or monitoring.

However, existing approaches have required the CF to be externally attached to the users' clothing or other substrates through manual techniques like sewing or thermal welding. This makes the CF based sensors prone to error if they are misplaced from the specific location. Besides, the sensitivity of the sensors is also often limited by the homogeneous patch of overlying CF.

Besides sewing and thermal welding, embroidery techniques have also been used as an alternative to combine the e-textiles onto the textile substrate with other electronic components. Few attempts have been made to design a textile circuit board that integrates electronic components and conductive yarn using embroidery technique. Though the embroidery technique can be used to integrate circuit components through external integration into wearer's garment, these approaches provide minimal control over the substrate shape and properties (stretchability, breathability etc.), which is often limited by the underlying garment.

To overcome this limitation, various example embodiments of the invention provide soft wearables with seamlessly integrated textile strain sensors, resistors and interconnects formed as a single holistic knitted piece. As discussed, the desirable electrical properties of a sensor, resistor and an interconnect are vastly contrasting, and this is achieved in a single conductive knit by locally varying the macroscopic electrical property of the conductive knit, through combined variations of the underlying yarn at the micro scale and the stitch pattern at the meso scale. Through suitable arrangement of the yarn and stitch types in the unit cell, electrical components such as interconnects and resistors that demonstrate negligible change in electrical resistance in response to strain were knitted. The strain sensors, resistors and interconnects were then distributed and knitted as a single holistic piece in a soft wearable knee brace for joint motion sensing in various example embodiments. It is understood that a range of other integrated soft wearables which can be used for monitoring of physiological signals, posture tracking, detection of speech disorders or gesture tracking, to mention a few among the plethora of several other potential applications may be obtained according to various example embodiments. The textile based soft wearables may be used for a variety of sensing purposes including joint motion sensing for sports and rehabilitation, prognosis and tracking of mobility healthcare conditions in the elderly, monitoring of physiological signals (e.g., heart rate, respiration rate), haptic feedback for gesture recognition that can be used in gaming, sign language, performing art and dancing. The soft wearables may be fabric based and the yarn materials used in their fabrication should be compatible with the knitting process. In various example embodiments, polymer based materials may be used for development of strain sensors.

Accordingly, various example embodiments provide a highly stretchable electrically conductive textile through the mechanical coupling of an electrically conductive and a dielectric yarn of high elasticity (ratio of Young's modulus of conductive yarn (E_{c} of about 1535 MPa) to spandex yarn (E_{d} of about 225 KPa), E_{c}/E_{d}>10³) in a stitch pattern in which the super elastic dielectric yarn (>440% strain) stretches significantly compared to the conductive yarn in the stitch pattern while also functioning to control the electrical contact points in the conductive yarn in the following manner: the super elastic dielectric yarn enables periodic formation and breaking of contact points within the conductive yarn during stretching of the textile, for example, in a highly sensitive and highly stretchable strain sensor (>8 gauge factor, >150% strain); the super elastic dielectric yarn prevents the formation of contact points in the conductive yarn during stretching of the textile, for example, in a highly stretchable resistor (>250% strain), and/or the super elastic dielectric yarn preserves the original contact points in the conductive yarn during stretching of the textile, for example, in a highly stretchable interconnect (>100% strain).

### Effect of Stitch Pattern on the Electrical Properties of Wale-Wise Knitted Strain Sensors and Interconnects

Using different stitch patterns can allow a single conductive yarn to take on varying electrical properties and serve different functions in an electrical circuit. However, the relationship is not so well understood due to the vast variety of stitch patterns available. Various example embodiments seek to contribute to this understanding by characterizing thirteen basic stitch patterns with potential for strain sensing. It has been found that changing the stitch pattern increased the gauge factor (sensitivity) by 7.16 times, the working range by 2.90 times, and the resistance by 3.50 times. In various example embodiments, Rib 1×1 and Interlock x2 may be used for strain sensor with high sensitivity and working ranges, while Linen Stitch and Full Cardigan may be used for interconnect with low sensitivity. Various example embodiments provide wearable sensors for human motion sensing.

Knitted sensors are gaining traction because they are flexible, can contain multiple yarn materials and knit structures, and can adopt the texture and appearance of regular garments. This makes knitted sensors ideal for fabricating comfortable and unobtrusive wearable sensors for long-term healthcare monitoring.

Machine knitted fabrics are made on knitting machines with rows of needles (needle beds) that form yarn into interlocking knitted loops. The three basic stitch types (knit, tuck, miss) and stitch transfer actions can be combined to form stitch patterns, which are modules of knitting instructions that repeat periodically to form a knitted fabric. Prior research shows that varying the stitch pattern can change the fabric's electrical properties by changing the interlocking loops' linear resistance, contact resistance, and response to strain.

Considering that there exist thousands of stitch patterns, it may be possible to use a single type of conductive yarn to knit different electrical components in a circuit. However, most papers only examine a few stitch patterns. Therefore, to broaden our understanding on the relationship between stitch patterns and electrical performance, thirteen basic stitch patterns are compared to select candidates for knitted strain sensors and interconnects, with a long-term goal of knitting a wearable strain sensor circuit for human joint kinematic applications.

### Method

### Knitted Conductive Samples

A Shima Seiki MACH2XS153 WHOLEGARMENT® 15 gauge flat-bed weft knitting machine with four needle beds (two lower beds and two upper beds) was used to knit three samples for each stitch pattern, resulting in a total of 39 samples. Of the 13 stitch patterns, nine are single bed stitch patterns that only require one lower needle bed, while four are double bed stitch patterns that require loops to be knitted on both lower needle beds simultaneously. All of the samples were knitted as wale-wise (vertically) oriented rectangular conductive patches measuring 6 wales (columns)×88 courses (rows), apart from Interlock and Full Cardigan that had 44 courses instead. The conductive patches measured approximately 4.5-8.5 mm×58-80 mm before undergoing the cyclic strain tests.

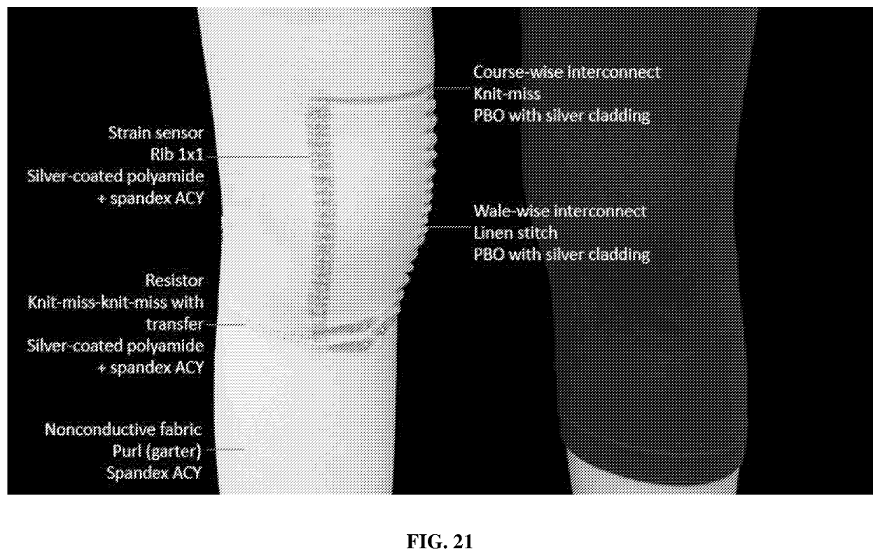

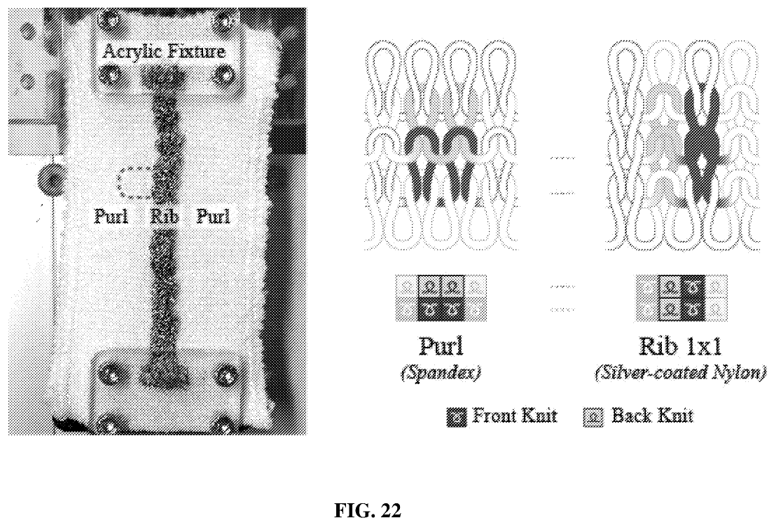

The electrically conductive patch was knitted with silvercoated polyamide yarn (Statex Shieldex® 235/36 dtex 2 ply HC+B). The conductive patches were surrounded by non-conductive spandex yarn (Zhejiang Kangjiesi air covered yarn, 100D spandex with 300D polyester DTY) knitted using purl (garter) stitch pattern, as illustrated in FIG. 22. More particularly, FIG. 22 shows an image illustrating a knitted conductive sample mounted on linear cyclic strain test setup (left) and close-up diagrams (right) of the knitted loops, where the surrounding spandex fabric is knitted with purl (garter) stitch pattern and the conductive patch is knitted with rib 1×1 stitch pattern.

### Linear Cyclic Strain Test Protocol

To understand the electromechanical properties of the knitted fabric under repeated stretching, each knitted sample underwent a linear cyclic strain test at 90% strain and 50 cycles. The experimental setup comprised a linear stage controlled using a stepper motor (Model AZMA69AK from Oriental Motor) and connected to an

Arduino Uno. The knitted sample was mounted using acrylic fixtures and a constant current of 30 mA was provided across the sample via connectivity pins using an Extech (382260)-80 W switching mode DC power supply. Subsequently, a triangular strain protocol with 90% strain amplitude and 3.33 cms-1 strain rate was programmed into the Arduino Uno and the sample was stretched along its wale-wise axis. The output voltage measured across the knitted sample and the encoder data from the stepper motor were recorded at 200 Hz (5 ms) using LabVIEW connected to an NI myRio real-time embedded evaluation board.

For analysis, the measured output voltage and encoder data were normalized according to the initial voltage V₀ and initial length L₀ of the sample. The normalized measured voltage is given by,

$${ε}_{V} = ( \frac{Δ V}{{V}_{O}} )$$

and the normalized length by,

$${ε}_{x} = ( \frac{Δ L}{{L}_{O}} ) ,$$

where ΔV and ΔL represent the change in voltage and change in length, respectively, of the knitted sample upon stretching.

### Response Variables and Selection Criteria

The electrical performance was evaluated based on:

### Gauge Factor (GF):

A dimensionless quantity that reflects the sensitivity of the knitted sample. It is defined as the ratio of the normalized voltage to normalized length, i.e.,

$$G F = ( \frac{Δ V / {V}_{O}}{Δ L / {L}_{O}} ) .$$

### Working Range (WR):

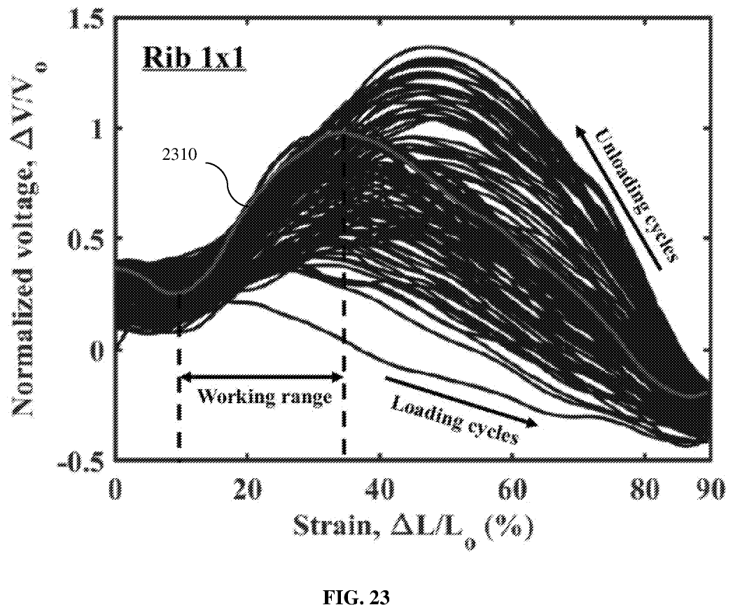

The practical range of the strain sensor that produces the maximum positive change in voltage (ΔV), i.e. the strain range in which the knitted sample is most sensitive. It is marked by two quantities, the start of the working range and the end of the working range, in %. FIG. 23 shows a voltage-strain graph for one Rib 1×1 sample showing how the working range is determined for a single loading cycle. The line 2310 is the 45th loading cycle. To compare the size of the working range, the difference between the start and end of the working range as expressed in percentage points (pp) was used. Both the gauge factor and the working range were calculated based on the loading cycle, i.e. as the knitted sample is increasing in length. Resistance: The opposition to the electrical current flowing in the conductive patch. The resistance measurements were taken before the cyclic strain test using an Agilent U1271A True RMS Multimeter. The resistance values were normalized by the conductive patch's relaxed length as measured before the cyclic strain test.

An ideal strain sensor should have a high gauge factor and large working range so that it can precisely sense a wide range of motion. An ideal interconnect should have a low gauge factor and low resistance so that it has negligible resistance regardless of strain. Ideally, both sensors and interconnects should perform consistently across the three samples.

### Experimental Results

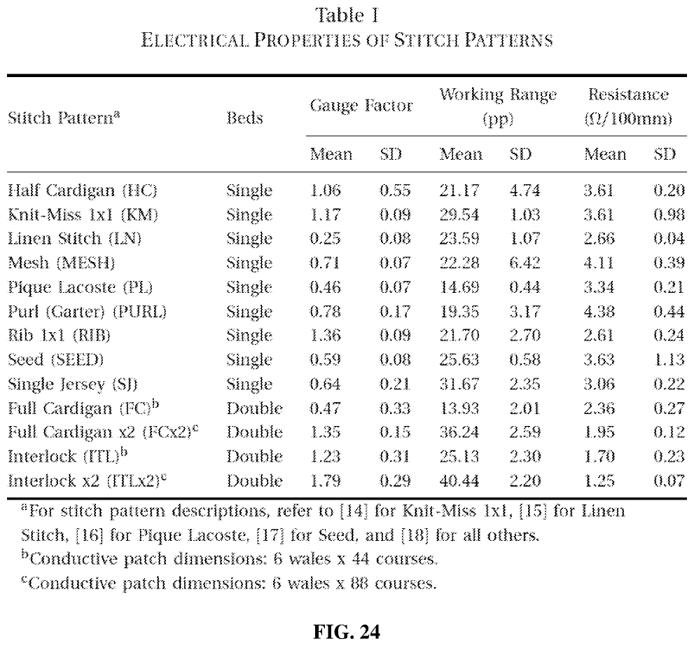

FIG. 24 depicts a Table I showing the means and standard deviations of the gauge factor, working range, and resistance measurements for each stitch pattern.

### A. Gauge Factor

For single bed stitch patterns, Rib 1×1 (GF: 1.36) and Knit-Miss 1×1 (1.17) had the highest GFs, while Linen Stitch (0.25) and Pique Lacoste (0.46) had the lowest GFs. For double bed stitch patterns, Interlock x2 (1.79) had the highest GF, while Full Cardigan (0.47) had the lowest GF.

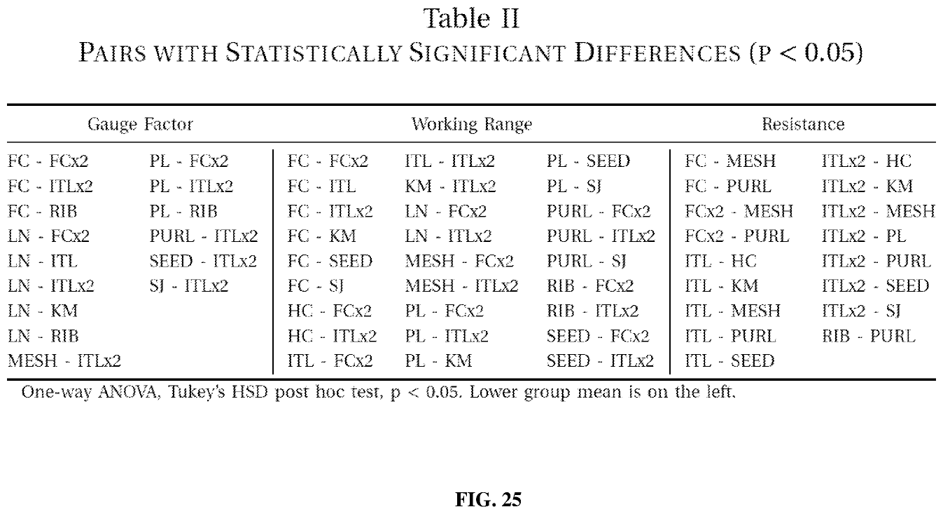

Comparing Linen Stitch and Interlock x2, the latter's GF was 7.16 times higher. There were statistically significant differences between group means as determined by one-way ANOVA (F(12,26)=7.210, p=1.377×10−5). To check specifically which group means have statistically significant differences, Tukey's HSD post hoc test was used to compare all pairs of group means while controlling the experiment-wise error rate. The pairwise comparison results are presented in Table II as shown in FIG. 25.

Comparing to other studies on wale-wise weft knitted fabric strain sensors, Xie et al. “High sensitivity knitted fabric strain sensors,” Smart Materials and Structures, vol. 25, pp. 1-7, 2016, tested single jersey samples. Their silver-coated nylon yarn sample had a very low GF of 0.05 (0-30% strain range). However, their cotton-stainless steel (SS) yarn sample had GF of −20 (0-5% strain range) and −1.52 (5-40% strain range). Similarly, Ehrmann et al. “Suitability of knitted fabrics as elongation sensors subject to structure, stitch dimension and elongation direction,” Textile Research Journal, 84(18), pp. 2006-2012, 2014, used polyester-SS yarn and their full cardigan sample had a GF of −8 (0-10% strain range). The higher absolute GFs and negative GFs are because the yarns are a blend of conductive (SS) and non-conductive fibers. Unlike silver-coated yarns which have a small yarn-to-yarn contact resistance, SS fiber blended yarns have a high yarn-to-yarn contact resistance due to the random distribution of conductive fibers. Under tension, the yarn-to-yarn contact force and contact area increase, making more conductive fibers touch each other. Thus, the resistance drops sharply at the start of the strain range.

### B. Working Range

For single bed stitch patterns, Single Jersey (WR: 31.67pp) and Knit-Miss 1×1 (29.54 pp) had the largest WRs, while Pique Lacoste (14.69 pp) and Purl (19.35 pp) had the smallest WRs. For double bed stitch patterns, Interlock x2 (40.44 pp) had the largest WR while Full Cardigan (13.93 pp) had the smallest WR.

Comparing Full Cardigan and Interlock x2, the latter's WR was 2.90 times larger. There were statistically significant differences between group means as determined by one-way ANOVA (F(12,26)=14.271, p=1.618×10⁻⁸).

### C. Resistance

For single bed stitch patterns, Purl (4.38Ω/100 mm) and Mesh (4.11Ω/100 mm) had the highest resistances while Rib 1×1 (2.61Ω/100 mm) and Linen Stitch (2.66Ω/100 mm) had the lowest resistances. For double bed stitch patterns, Full Cardigan (2.36Ω/100 mm) had the highest resistance, while Interlock x2 (1.25Ω/100 mm) had the lowest resistance. All of the double bed stitch patterns had lower resistances than the single bed stitch patterns.

Comparing Interlock x2 and Purl, the latter's resistance was 3.50 times higher. There were statistically significant differences between group means as determined by one-way ANOVA (F(12,26)=8.101, p=4.754×10⁻⁶) (Table II in FIG. 25).

### D. Strain Sensor Candidates

For single bed stitch patterns, Rib 1×1 is the best strain sensor candidate because it had the second highest sensitivity and a moderate working range. An alternative candidate is Knit-Miss 1×1, which has a lower sensitivity but a larger and more consistent working range.

For double bed stitch patterns, Interlock x2 is the best candidate because it had the highest gauge factor and largest working range, although it has moderate consistency between samples. An alternative candidate is Full Cardigan x2, which had the third highest gauge factor and second largest working range.

### E. Interconnect Candidates

For single bed stitch patterns, Linen Stitch is the best interconnect candidate because it had the lowest gauge factor, which means that its resistance will not change much when stretched. It also had the second lowest resistance among the single bed stitch patterns.

For double bed stitch patterns, Full Cardigan is the best candidate because it had the third lowest gauge factor, although it also had the highest resistance among the double bed stitch patterns.

Due to practical limitations, only three samples were tested for each stitch pattern. Also, the conductive patches were standardized by number of courses and wales, and therefore had varying dimensions. The measurements were normalized by length to compensate for differences in length, but the varying widths and thicknesses of the knitted patches are expected to affect the results too.

Various example embodiments demonstrate that using different stitch patterns can substantially change the electrical performance of knitted conductive fabrics, allowing a single conductive yarn to take on different properties and different electrical functions depending on the stitch pattern.

Knitted Wearables with Embedded Sensors of Tuneable Sensitivity

There has been tremendous interest in the field of e-textiles for the development of soft wearables. Most of the existing textile-based wearables rely on integrating off-the-shelf commercial conductive fabrics to a substrate through sewing or thermal welding. As a result, the sensors' sensitivity and working range is often limited by the overlying conductive fabric. In various example embodiments, both the sensitivity and the working range of the sensors may be tuned by creating the conductive fabric from scratch and varying the knit structure by controlling various knitting parameters, for example, loop length, stitch pattern or the arrangement of different yarn materials. Through the distribution of sensors of desirable sensitivity and working range within a soft wearable, for example a knee brace, various example embodiments provide a novel approach to sense multiple attributes, such as multiple degrees of motion, knee alignment or radius of the knee. Moreover, these sensors may be connected through well distributed interconnects which together with the sensors may be seamlessly integrated into the soft wearables, forming a truly embedded network of sensors and interconnects within a single piece of knitted soft wearable. Owing to the stretchable, breathable, lightweight, and soft texture of the textiles, they can be used for long-term continuous monitoring of joint kinematic movements for prognosis, mobility condition tracking or rehabilitation monitoring. Various example embodiments may be used for motion sensing (e.g., human joints or other moving parts), monitoring of physiological signals (e.g., respiration and heart rate), the prognosis of healthcare conditions, disease trajectory tracking, rehabilitation, posture tracking, sensing of speech disorders.

Long-term continuous monitoring is vital to track various healthcare conditions in human populations. This is enabled by a range of wearables that are commercially available. However, most of these wearable devices are made of rigid materials such as metals and plastics. In recent times, there has been tremendous interest in e-textiles for the development of soft wearables. Owing to their stretchability, breathability, light weight and soft feel on skin, they serve as an excellent material choice for the development of soft robotics and soft sensors that can be integrated into daily knitwear. Some of the fabric-based wearables have been proposed in literature for respiratory monitoring, joint motion sensing, etc. One of the major drawbacks of these fabric-based wearables is that they either rely on commercially available conductive fabric which is manually sewn to the wearer's external garments or fabricated using manual drop casting methods, thus greatly limiting their scalability. Besides, the sensitivity of the sensors is often limited by the overlying conductive fabric.

To overcome this limitation, various example embodiments provide tuneable sensors from scratch by varying the geometry, pattern and material of the knitted yarn forming the conductive fabric, using a Computer Numerical Control (CNC) knitting machine. The CNC knitting machine is analogous to a multi-material 3D printer with the ability to integrate various stitch patterns, stitch densities and yarn materials seamlessly into a single textile with minimal post-processing. Most of the previous works on soft knitted sensors have focused on either developing electromechanical models to predict the resistance or investigating the effect of knitting parameters on sensitivity of the sensors. However, there has been limited work on the development of truly embedded soft knitted wearables for long term monitoring. Through the distribution of sensors of desirable sensitivity and working range, various example embodiments provide wearables that can be used to sense multiple attributes. Moreover, these sensors can be connected through well distributed interconnects which together with the sensors can be seamlessly integrated into the soft wearables, forming a truly embedded network of sensors and interconnects within a single piece of knitted soft wearable. The tuneable nature of the knitted sensors are demonstrated and develop a seamlessly integrated soft knitted knee brace is provided that can be used for human joint sensing for prognosis, tracking or rehabilitation monitoring of mobility conditions.

The soft knitted knee brace can be used for long term continuous monitoring of mobility related conditions. For healthy individuals, continuous monitoring can be used for real-time activity tracking such as during sports activities. Also, it may facilitate detection of unusual patterns that do not conform to expected behaviours and raise warnings before an adverse event occurs. For the elderly, long-term continuous monitoring of human joints could aid greatly in tracking the onset and evolution of conditions such as sarcopenia, late-onset Pompe disease (LOPD), early stages of Parkinson's disease or osteoarthritis that cause mobility challenges and, in some cases, disability. LOPD is associated with a slow reduction of muscle strength, sarcopenia refers to the reduction of muscle mass as a result of ageing and osteoarthritis is caused by the gradual wearing down of the cartilage that cushions the bones. As these conditions evolve over time, joint motion data can help track the progression of these mobility related healthcare conditions which can be useful in aiding decisions necessary for clinical interventions.

The fabric-based soft knitted knee brace allow for reliable joint monitoring over long periods. In addition, various example embodiments enable a range of other soft wearables which can be used for monitoring physiological signals, posture tracking or detection of speech disorders.

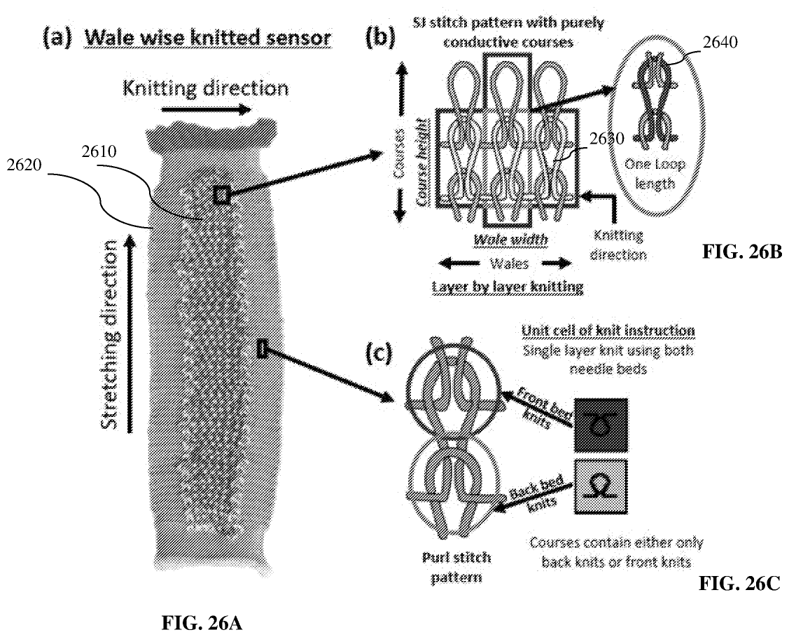

Enabled by the freedom to design knitted fabrics with a wide range of knit structures and yarn materials, sensors with tuneable sensitivity and working range can be created using CNC knitting machine, suited for desired functionality. FIGS. 26A-26C show schematics of a wale wise knitted sensor, single jersey stitch pattern of the conductive fabric, and purl stitch pattern of the surrounding fabric, respectively. FIG. 26A shows the schematic of a wale wise knitted sensor which is designed to stretch orthogonally to the knit direction. It includes the conductive sensor 2610 (in the middle) surrounded by a passive elastic fabric 2620 knitted using spandex yarn according to various example embodiments. The spandex yarn is chosen for the surrounding material to enhance the stretchability of the sensor strip. FIG. 26B shows the zoomed in view of the interlocked stitch pattern of the conductive fabric. A single row of the conductive fabric 2630 is called a course and measured in terms of course height. A single column is called a wale and is measured by the wale width. A simple single jersey (SJ) stitch pattern consisting of conductive courses is used for illustration. The amount of material used to knit a single loop 2640 is called the loop length as shown in FIG. 26B. FIG. 26C shows the stitch pattern of the surrounding elastic fabric. A purl stitch pattern is used for the surrounding fabric. The knitting instruction unit cell for a purl stitch pattern is also shown in FIG. 26C. Compared to a single jersey stitch pattern which consists of only front bed knits, purl stitch pattern consists of loops knitted alternately on front bed and back bed. A single jersey knit curls at the edges due to the deformation of the loops knitted on the front bed, whereas a purl knit remains flat because it is balanced by the opposing nature of the curls from the alternating courses of loops knitted on the front bed and back bed. Therefore, a purl stitch pattern may be used for the surrounding fabric.

The conductive nylon yarn employed shows negligible stretchability by itself and therefore demonstrates a limited change in resistance when the yarn alone is stretched. However, the sensor demonstrates high stretchability and high resistance change due to the stretchable geometry of the loop structure which causes a change in contact resistance. As the sensor is stretched, the loops pull each other thereby elongating the loops and reconfiguring the contact points. In the stitch pattern of the conductive sensor, the contacts happen first, between the adjacent loops of the same course and second, between loops of the adjacent courses which are interlocked with each other.

According to the Holm' s contact theory as described in Holm. R., 2013. Electric contacts: theory and application. Springer Science & Business Media, the contact resistance between two conductive electrodes is inversely proportional to the number of contact points and the contact pressure. As the sensor is stretched the adjacent loops of the same course first move away from each other resulting in a drop in contact pressure and eventually breaking of the contact points, thus increasing the contact resistance. Further stretching of the sensor results in the loops getting longer and narrower and as a result they start to move closer to each other, thus decreasing the contact resistance. Therefore, in a wale wise knitted sensor two distinct working regions may be observed.

### Tuneable Sensitivity and Working Range

Through the control of geometry of knitted loops, design of stitch pattern and the arrangement of different yarn materials, the sensitivity and working range of the sensors may be tuned. Moreover, these sensors may be seamlessly and readily integrated into daily knitwear. In this section, the above knitting parameters are varied and the performance of the sensors are evaluated on two metrics, their sensitivity measured by the gauge factor and their working range represented by the monotonic increase in resistance. The gauge factor is a dimensionless quantity given by,

$$GF = \frac{( {V}_{i} - {V}_{O} ) / {V}_{O}}{( {L}_{i} - {L}_{O} ) / {L}_{O}} ,$$

which is the ratio of the normalized voltage response to the normalized length whereas, the working range is measured as the strain of the sensor.
a) Loop Length Varying the loop length results in a change in the aspect ratio of the knitted

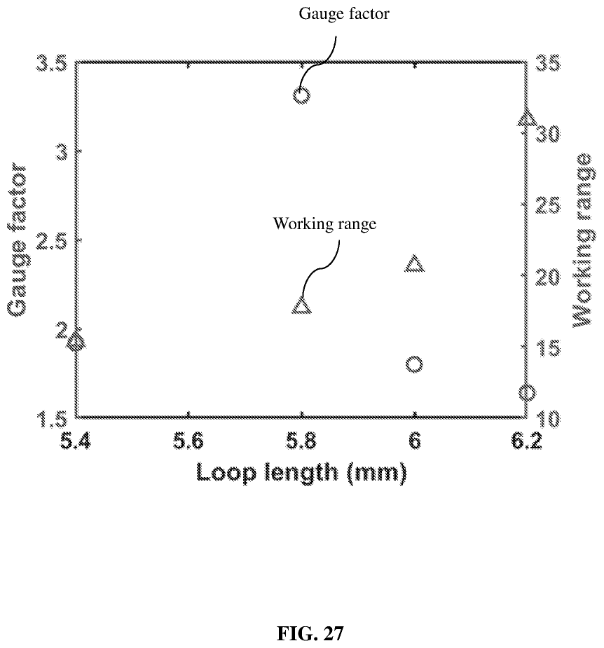

loops which in turn effects the number of contact points and contact pressure, thereby affecting their sensitivity. FIG. 27 shows the effect of loop length on gauge factor and working range of the sensors. The gauge factor initially increases with loop length before decreasing. Thus, there exists an optimum loop length that provides an enhanced contact area between adjacent loops and thereby providing increased sensitivity.

### b) Stitch Patterns

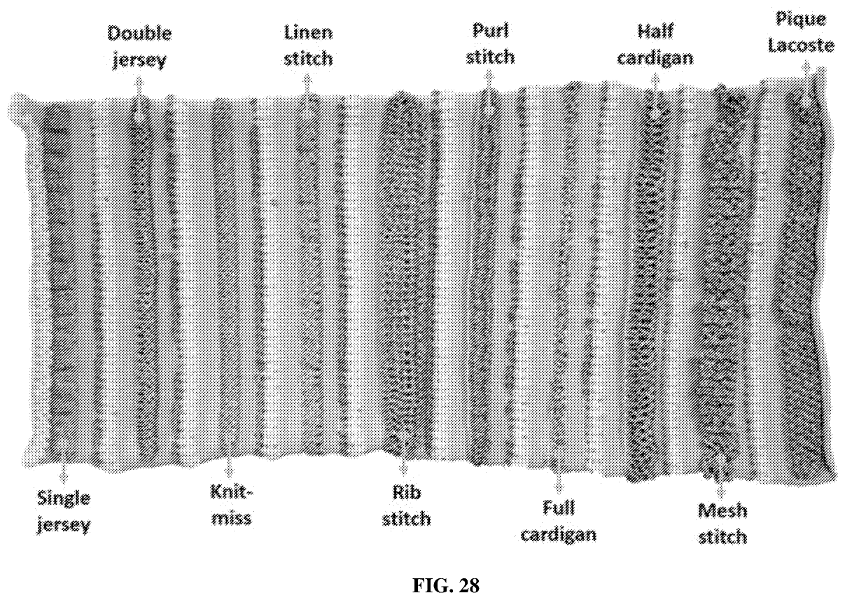

Different stitch patterns can be obtained by varying the distribution of knits, miss and tucks in the knitted structure, which in turn determines the working range and sensitivity of the sensors. FIG. 28 shows the knitted sensors of various stitch patterns. The design of different stitch pattern and their effect on wale wise knitted sensors of all conductive courses are demonstrated.

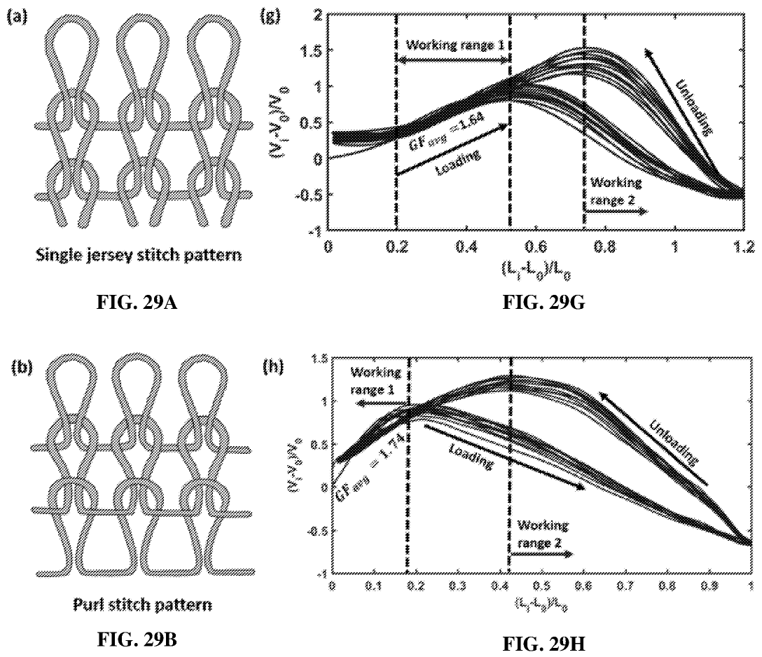

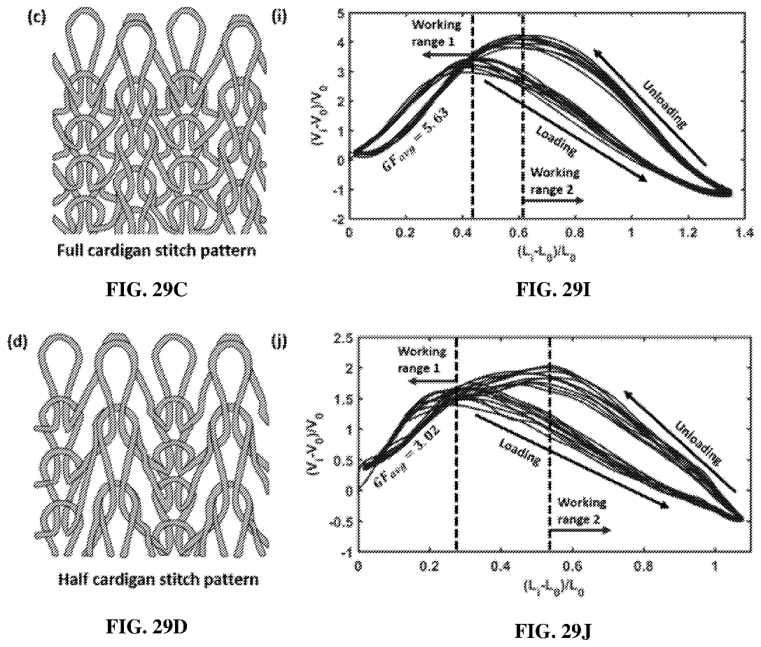

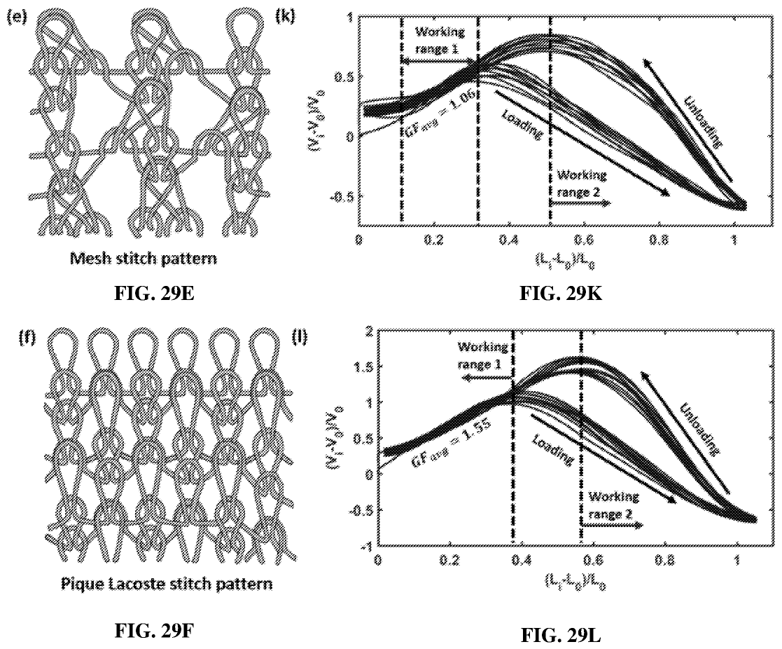

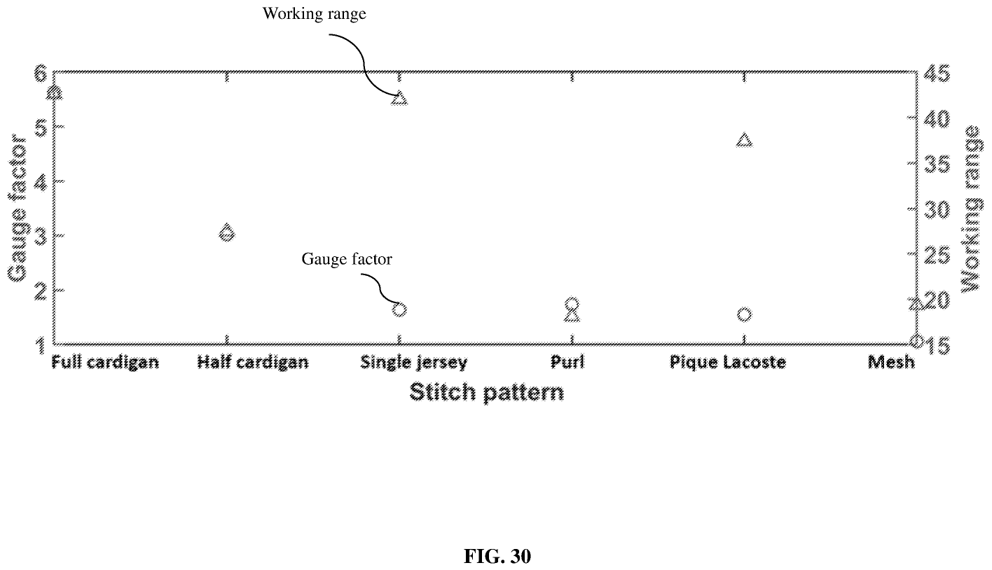

FIGS. 29A-29F show knitted sensors and their stitch patterns, while FIGS. 29G-29L show normalized voltage vs strain response of the respective sensors.

FIG. 29A shows the stitch pattern of a single jersey knitted sensor. It is the simplest stitch type, formed using only front knit stitches. FIG. 29G shows the normalized voltage vs strain response of the single jersey knitted sensor. Symbols V₀ and L₀ refers to the reference voltage and length at the rest state, respectively. The working range 1 (WR1-30.95%) is characterized by a monotonic increase in resistance during both loading and unloading trajectory whereas working range 2 (WR2) is characterized by a monotonic decrease during both loading and unloading trajectory. Moreover, the average gauge factor (1.64) is calculated for the loading curve of WR1 by taking an average of the gauge factor over 30 cycles.

FIG. 29B shows the stitch pattern of a purl knitted sensor. Purl stitch is formed by alternating courses of front and back knit stitches. FIG. 29H shows the normalized voltage vs strain response of the knitted sensor. Compared to the single jersey stitch pattern, the purl stitch offers a higher sensitivity with gauge factor of 1.74, but a smaller WR1 (18.1%). Additionally, its response is characterized by a smaller hysteresis loop (loading and unloading trajectory in WR1), simplifying its response.

Full cardigan (or polka rib) has one course of loops knitted on the front bed and tucks on the back, and the second course with the sequence reversed, as shown in FIG. 29C. This produces a balanced 1×1 tuck rib structure. Due to the alternating knit-tuck structure, this stitch pattern is looser than most other double bed patterns and is very stretchy. FIG. 29I shows the normalized voltage vs strain response of the knitted sensor. The average gauge factor for 30 cycles of a full cardigan knitted sensor is significantly higher at 5.63 with a WR1 of 42.57%. As can be seen, the hysteresis is larger for the full cardigan knitted sensor.

Half cardigan (or royal rib) has tucked loops on only one bed on alternate courses, as shown in FIG. 29D. On the all-knit side, one course of loops has very large and rounded loops because they receive yarn from the tuck loops on the other side. The other course of loops in contrast has extremely small and insignificant loops because they are robbed of yarn by the elongated held loops on the other side. This pattern is less elastic than full cardigan. FIG. 29J shows the normalized voltage vs strain response of the knitted sensor. The average gauge factor for 30 cycles of a half cardigan knitted sensor is smaller than a full cardigan at 3.02 with a WR1 of 27.44%.

Mesh stitches are formed by transferring and racking selected loops to move the loops to the left or right, typically so that some needles are empty while other needles hold multiple loops, as shown in FIG. 29E. The empty needles result in small holes in the fabric. FIG. 29K shows the normalized voltage vs strain response of the knitted sensor. The average gauge factor for 30 cycles of a mesh knitted sensor is 1.06 with a WR1 of 19.36%. As can be seen, the hysteresis loop is small for a mesh knitted sensor.

Pique lacoste is formed by alternating rows of knit-and-tuck stitches and rows of knit-only stitches, as shown in FIG. 29F. FIG. 29L shows the normalized voltage vs strain response of the knitted sensor. The average gauge factor for 30 cycles of a pique lacoste knitted sensor is 1.55 with a WR1 of 37.39%. The hysteresis loop is small for the pique lacoste knitted sensor.

FIG. 30 shows the comparison amongst the knitted sensors of various stitch patterns. More particularly, FIG. 30 shows the comparison of the gauge factor and working range of the sensors knitted with various stitch patterns. Full cardigan has the highest sensitivity followed by half cardigan. Also, full cardigan, single jersey and pique lacoste offer a significantly larger working range. Besides, as discussed above, the full cardigan, half cardigan and single jersey stitch pattern show significant hysteresis in their WR1. On the other side, purl, pique lacoste and mesh stitch patterns show smaller hysteresis in WR1 which simplifies development of models to predict their response.

### c) Alternate Conductive Courses

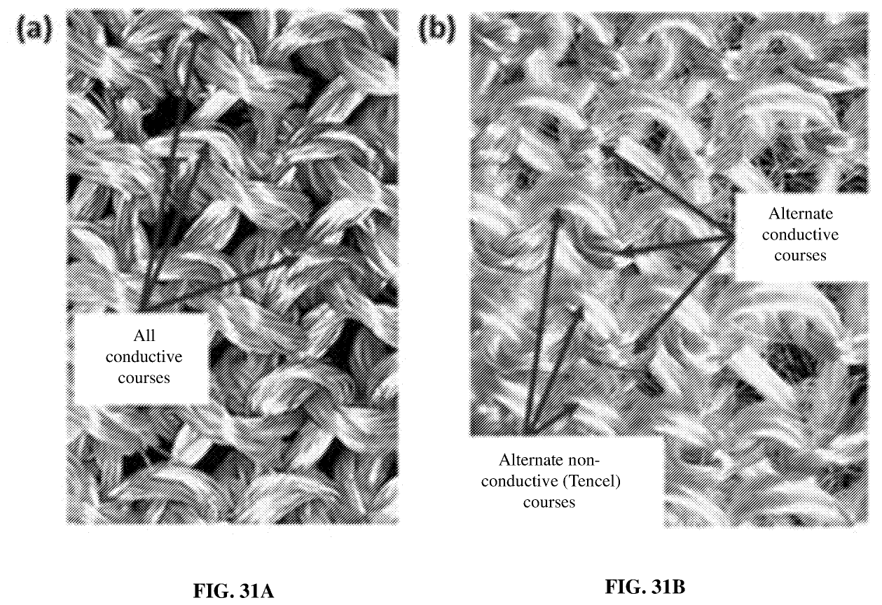

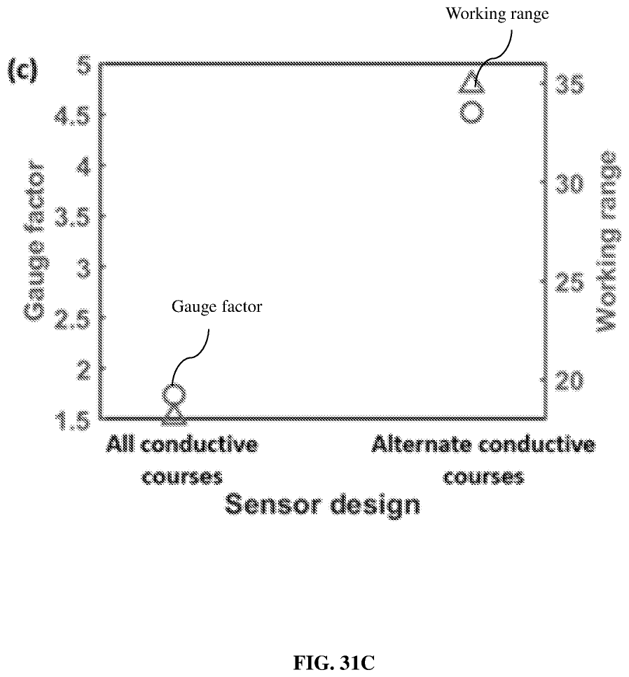

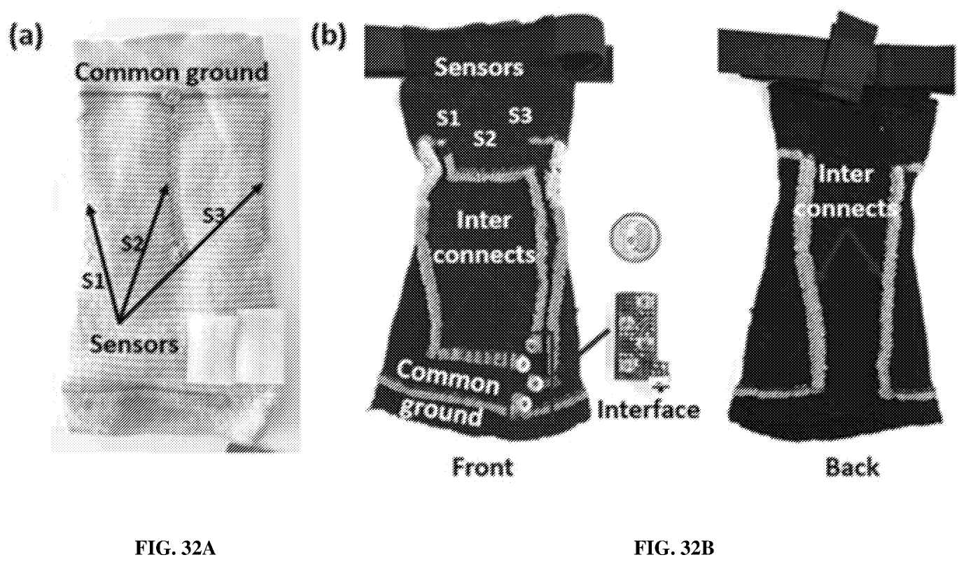

In contrast to all conductive course sensor, a sensor with alternate conductive courses is provided. In various example embodiments, purl stitch pattern was employed, owing to its simple structure. FIGS. 31A-31B show images of the design of all conductive courses sensor and design of alternate conductive courses sensor, respectively. Compared to all conductive course sensor shown in FIG. 31A, the alternate conductive courses sensor has courses of conductive yarn separated by non-conductive Tencel yarn as shown in FIG. 31B. For example, cellulose fibre-based Siro Spun 15/1 yarn made from Tencel is used for knitting the non-conductive courses. FIG. 31C show a graph illustrating a comparison of performance between all conductive and alternate conductive courses sensors. As can be seen in FIG. 31C, the gauge factor and working range of the alternate conductive course sensor is significantly higher than all conductive course sensor. This is because when an alternate conductive course sensor is stretched, the non-conductive courses separate the conductive courses, thus increasing the resistance. In comparison, in an all conductive course sensor, the conductive loops in adjacent courses are interlocked. As all conductive course sensor is stretched, the loops that touch each other within the same course tend to move farther apart thus increasing the resistance according to the Holm's contact theory. At the same time, the adjacent courses that are interlocked tend to pull each other, thus increasing the contact area and the contact pressure, resulting in a decrease in resistance. The result of these two competing factors lower the change in resistance of an all conductive course sensor and hence its sensitivity.

### Soft Knitted Knee Brace

A soft knitted knee brace with sensors and interconnects seamlessly integrated and distributed within the soft wearable is provided according to various example embodiments. Sensors of desirable sensitivity and working range may be distributed to sense multiple knee joint attributes, such as multiple degrees of motion, knee alignment or radius of the knee. Moreover, these sensors are connected through well distributed interconnects, which together with the sensors form a truly embedded network of sensors and interconnects within a single piece of knitted soft wearable. A high level of personalization may be obtained and the soft wearable may be designed to precisely fit the wearer's geometry. Moreover, the distribution and placement of sensors may be highly customized to suit different functionalities.

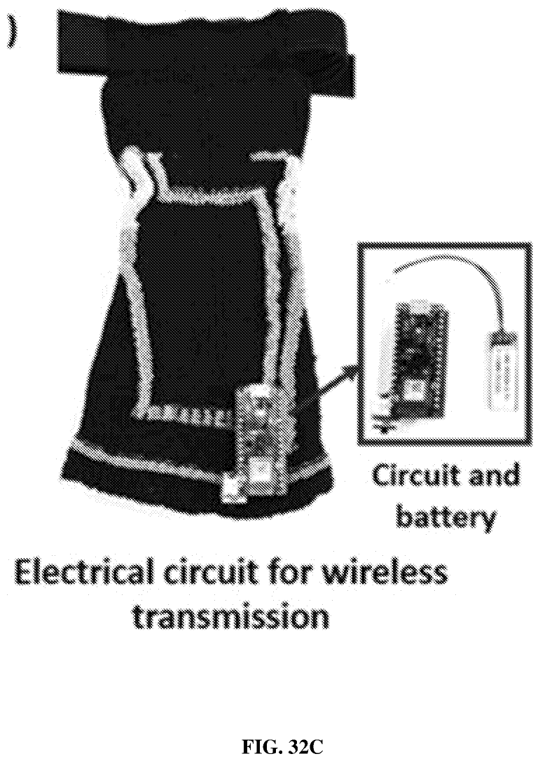

FIGS. 32A-32B show images of knee braces according to various example embodiments. More particularly, FIG. 32A shows the knee brace integrated with three sensors S1, S2 and S3 and the interconnect forming a common ground at the top according to various example embodiments. The purl stitch pattern with alternate conductive courses was chosen as the sensor pattern due to its high sensitivity and working range. The sensors sit on the centre and sides of the knee's patella region to track the knee joint's alignment in real-time and the degree of flexion-extension the knee undergoes. Comparing the reading of the sensor S1 and S3 facilitates in determining if the knee is aligned inwards or outwards, which causes the non-homogeneous distribution of pressure on the joints, thus resulting in its differential wear and tear.

FIG. 32B shows another knee brace in which the sensors are distributed along the circumference of the knee (S2), in addition to the sides of the patella region (S1 and S3). The course wise sensor S2 is knitted in half cardigan stitch pattern due to its high sensitivity and working range and allows for measurement of variation in radius of the knee during routine activity. The collected electrical signal can be processed into the arc length and using the angle (θ) from the design allocated point in the knee brace, the radius of the users' knee may be estimated.

Similarly, sensors may be distributed on other regions of the knee brace to pick up other degrees of motion such as adduction-abduction, internal-external rotation in addition to flexion-extension. These additional angle measurements can be captured and related by finding the optimal sensor placement using a four-bar linkage design mechanism or a parallel manipulator platform.

FIG. 32C shows a knee brace with electrical circuit for wireless transmission. An Arduino Nano board powered by a 3.7V small Li-Po battery is employed to record the voltage across the knee brace sensors. Metallic buttons are attached to the outside of the knee brace to connect the ends of the sensors and the common ground to the circuit, as shown in FIG. 32C. Tiny pieces of Velcro are stitched to the knee brace (bottom right) to mount the circuit board and prevent it from falling off. The data collected through the sensors can then be transmitted wirelessly to a smart device such as a smartphone or a tablet. The soft knitted knee brace can thus be used for long term continuous monitoring of joint kinematic movements.

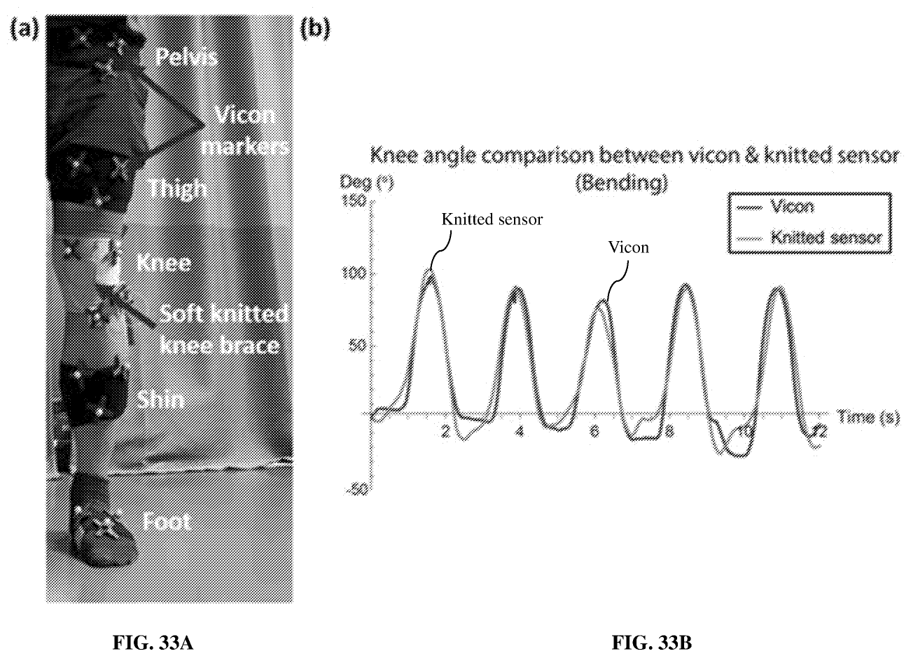

To verify the knee brace measurement accuracy, a Vicon (Oxford, UK) system was utilized as the reference for comparison. A total of 15 reflective markers were attached to the subject's lower left extremity, and a flexion-extension activity was recorded on nine motion-capture cameras and by the soft knitted knee brace, as shown in FIG. 33A. More particularly, FIG. 33A shows a soft knitted knee brace and the distribution of markers for Vicon motion capture system according to various example embodiments. The data sets were processed in MATHEMATICA, and the knee angle motion along the sagittal plane was compared. The 15 reflective markers provide the coordinates of the markers in space thus enabling to calculate the centre of rotation (COR) and the axis. The electrical signals were processed using a pulley model system to relate to the knee angular motion. First, the strains are calculated using the polynomial function model. Next, using the calculated strain and the subject's anthropometric radius, the knee angular motion may be obtained using the equation L=Rθ along the sagittal plane during the flexion-extension cycles. FIG. 33B shows the comparison between knee angle measured by the Vicon system versus the soft knitted knee brace according to various example embodiments, for several cycles of flexion-extension. The result shows that the soft knitted knee brace according to various example embodiments performs closely to the Vicon measurement system and can therefore be used to effectively monitor and record the knee motion along the sagittal plane.

Besides joint motion sensing, various example embodiments may also enable a range of other soft wearables which may be used for long term continuous monitoring of physiological signals, posture tracking or detection of speech disorders.

### Differential Course Design for Mechanical Pre-Strain

The fabric-based sensors show two distinct working regions, WR1 and WR2 as shown in FIGS. 29A-29L. While the WR1 can be realised by the sensor embedded in its rest state, to realise WR2 the sensors need to be pre-stretched by a certain amount. This is done by designing the sensor to be shorter than the surrounding fabric. This is achieved by reducing the number of courses in the sensor compared to the number of courses in the surrounding knit. Depending upon the difference in the number of courses, the sensor is stretched by the desired pre-strain.

### Non-Linear Modelling Using Deep Learning

Owing to highly non-linear behaviour of the underlying yarns and stitch patterns, the fabric-based sensors show significant non-linearities such as rate-dependent hysteresis coupled with drift and delay which makes it challenging to predict their response. To account for these behaviours, data driven models were developed using deep learning approaches. The developed models may effectively compensate for these non-linear behaviours which enables predicting the sensor response for the entire working range (WR1 or WR2) of the respective sensing regions.

For developing data-driven models, sensor response for different protocols were recorded by applying loading-unloading cycles of different stretching rates, cyclic stretching for thousands of cycles and various trajectory like staircase and step. A long-short term memory network (LSTM) recurrent network is trained on the recorded data to predict the linear or bending strain given the voltage response of the sensor. Once the model is trained, it is then verified by comparing the model response with the experimentally recorded data from the sensor for random strain inputs.

### Experimental Setup

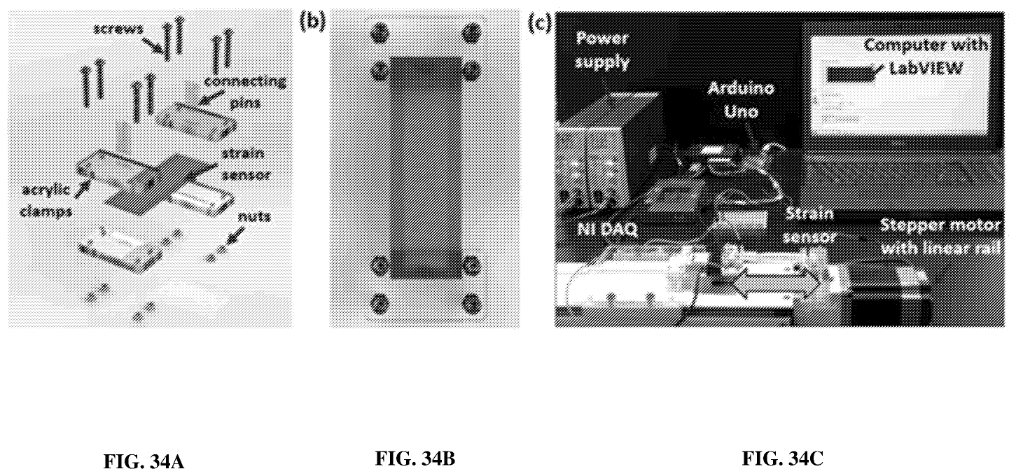

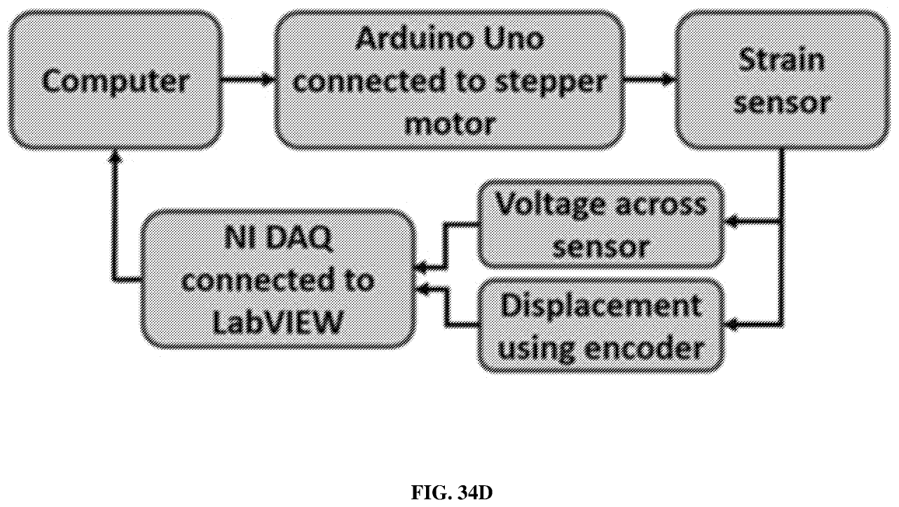

A customised experimental setup was developed to measure the resistance change across the strain sensor upon stretching. FIGS. 34A-34D show exploded view of the sensor mounting and connections, assembled sensor, experimental setup, and block diagram of the experimental setup, respectively. The interface for attaching the knitted sensor is shown in FIG. 34A, which includes two rectangular clamps to hold the knitted sensor. The clamp is made from 5 mm thick transparent acrylic, and the knitted sensor is then secured using four socket screws and five connectivity pins to prevent the knitted sensor from slipping during the test. A small constant current was supplied across the sensor to measure the electromechanical property, measuring the output voltage across the knitted sensor as its resistance changes under strain. FIG. 34B shows the assembled view of the sensor.

Next, the strain sensor is attached to the experimental setup which includes a linear stage to provide the necessary strain input using a stepper motor, as shown in FIG. 34C. The output voltage is measured across the knitted sensor along with the stepper motor's encoder data and recorded simultaneously at a sampling frequency of 200 Hz (5 ms) using a real-time embedded evaluation board (National Instrument: NI MyRio). Two Extech (382260)-80 W switching mode DC power supplies are also employed. The first power supply provides a constant current (e.g., 30 mA) across the knitted sensor and the second power supply provides constant voltage (e.g., 24 V) to the load cell. FIG. 34D shows the block diagram of the experimental setup.

All the tests were conducted using the experimental setup described above and the measured voltage and encoder data were normalised according to the reference voltage

$$v = \frac{( {V}_{i} - {V}_{O} )}{{V}_{O}}$$

and initial length

$$ε = \frac{( {L}_{i} - {L}_{O} )}{{L}_{O}} ,$$

where V₀ and L₀ denote the initial voltage and length of the fabric prior to strain, Vᵢ is the voltage, and Lᵢ is the length of the fabric at the stretched state.

Joint range of motion (ROM) analysis systems are classified into non-wearable systems (NWS) and wearable systems (WS). Motion capture system (Mo-cap) and ground force plate sensor are some of the classic examples of NWS, and these systems can provide reliable results. However, the measurements must be taken in a controlled laboratory environment and require trained personnel to operate the devices, making them unsuitable for long-term joint monitoring and sustainable rehabilitation treatment.

The inertial measurement unit (IMU) is the typical WS sensor used in joint motion sensing. However, to estimate the joint motion, a minimum of two IMUs are needed to sense the limb relative angular velocity and acceleration, which could obstruct the user's movement during the measurement. Apart from this, IMU has an unavoidable drift issue that requires additional computational work to achieve desirable motion data.

Wearable sensors made from conductive fabric (CF) or e-textiles have received growing interest for human joint sensing applications. These CFs can be designed into a piezoresistive sensor due to their highly elastic property, and they can exhibit intrinsic resistance change when they are stretched. These CF-based sensors have many advantages over existing NWS and IMU devices, as they are light weight, non-invasive and can be integrated into the garment, providing more comfort for the patient. These advantages enable a potential use of CF sensors for long-term joint motion monitoring.

However, existing approaches have required the CF to be externally attached to the users' clothing or other substrates through manual techniques like sewing or thermal welding. This makes the CF based sensors prone to error if they are misplaced from the specific location. Besides, the sensitivity and working range of the sensors are also often limited by the overlying CF. To overcome this limitation, soft wearables with seamlessly integrated and distributed network of strain sensors and interconnects using a CNC knitting machine that can be personalized to wearer's geometry are provided according to various example embodiments, allowing for reliable joint monitoring over long periods of time. Moreover, various example embodiments allow to tune both the sensitivity and the working range of the sensors by varying the structure of the knit through adjustment of knitting parameters such as loop length, stitch pattern and the arrangement of different yarn materials.

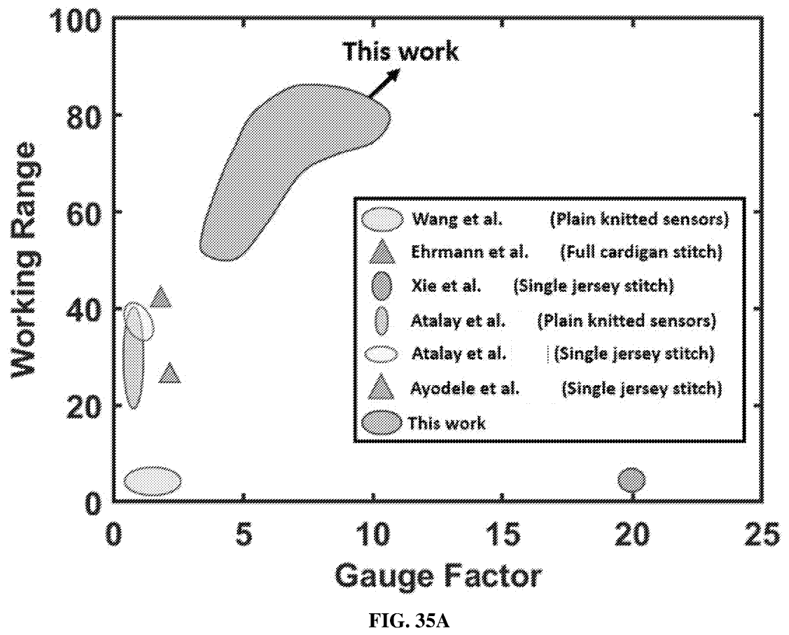

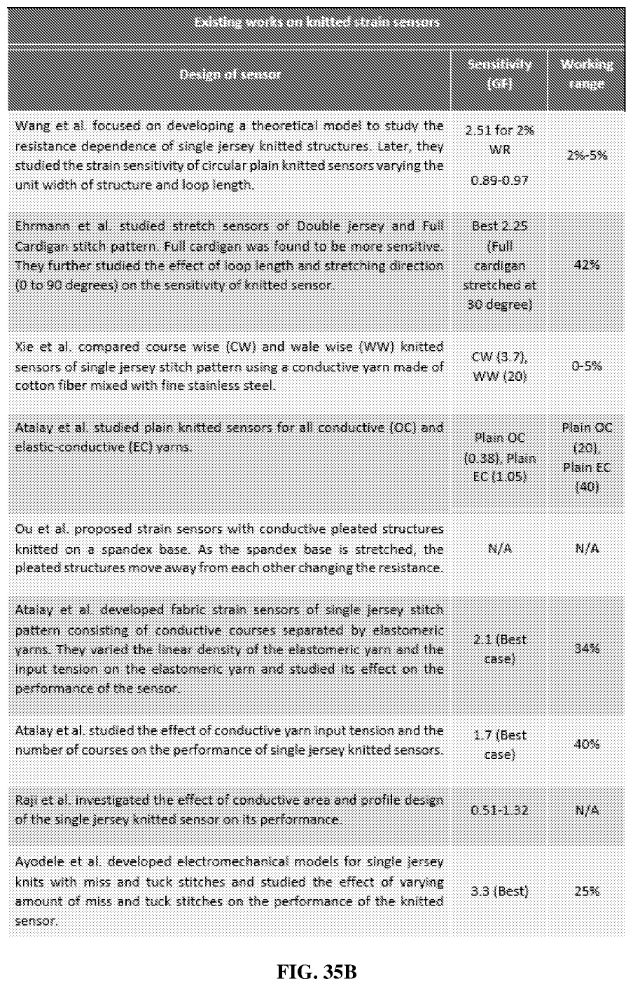

FIG. 35A shows a graph illustrating the comparison of the sensitivity and working range of the soft knitted sensors to literature, while FIG. 35B shows a table illustrating the sensitivity and working range of the literature.

Soft Wearable Knee Brace with Embedded Sensors for Knee Motion Monitoring

E-textiles have shown great potential for development of soft sensors to track human joints in applications such as sport & joint injury rehabilitation, soft robotics, and entertainment. However, existing approaches require the sensors to be attached externally onto the substrate or the garment. Various example embodiments address the issue by embedding the sensor directly into the wearable using a computer numerical control (CNC) machine. The capability to knit sensor with the stretchable surrounding fabric is demonstrated. Next, the sensor is characterized and a model for the sensor's electromechanical property is developed. Lastly, a fully knitted knee brace with embedded sensor is developed and tested by performing three different activities: Flexion-extension, walking, and jogging with a single test subject. Results show that the knitted knee brace sensor can track the subject's knee motion well, with a Spearman' s coefficient (rₛ) value of 0.87 when compared to the reference standard.

Long-term continuous monitoring of human joints can provide vital information that can be used to monitor recovery from sports injuries, stroke rehabilitation or even aid in the detection of early stage of Parkinson's disease. Although visual inspection is able to identify joint motion abnormalities, clinicians prefer to identify, locate, and monitor abnormalities using quantifiable and accurate measurement systems. Therefore, providing clinicians with crucial information on joint range of motion (ROM) may help clinicians to detect physical lesions that may affect the patients' activities of daily living (ADL) and provide early intervention for Parkinson's disease or rehabilitation.

The current standard for joint ROM analysis can be classified into the non-wearable systems (NWS) and wearable systems (WS). Motion capture (mo-cap) and ground force plate sensors are some of the classic examples of NWS that provide highly repeatable and reproducible results. However, the measurements are usually taken in a controlled laboratory environment and require trained personnel to operate the devices, making them impractical for long-term joint monitoring and sustained rehabilitation treatment.

Inertial measurement units (IMUs) are the typical WS used in joint motion sensing. However, to estimate the joint motion, a minimum of two IMUs are needed to sense the limb's relative angular velocity and acceleration, which could obstruct user's movement during measurement. Besides, IMUs have an unavoidable drift issue that requires additional computational work to achieve desirable motion data.

There has been tremendous interest in the development of soft wearable e-textiles. Owing to their stretchable, breathable, light weight and soft texture, they serve as an excellent material choice for the development of soft sensors and actuators that can be integrated into daily knitwear. In some works, commercially available conductive fabrics (CFs) are used to make piezoresistive sensors due to their highly elastic property and intrinsic resistance changes when undergoing strain. These CF-based sensors have many advantages over existing NWS and IMU devices as they are lightweight, non-invasive and unobtrusive. This makes them more convenient and comfortable for patients, and also more practical for long-term ROM monitoring.

However, most of the existing studies require the CF-based sensor to be attached to the user's clothing or substrate externally, making it prone to error if the sensor is displaced from its intended location. To overcome this limitation, various example embodiments provide a soft knitted garment with embedded sensors that can be knitted as a single piece using a computer numerical control (CNC) knitting machine.

### CNC Knitting Sensor & Characterization

### A. CNC Knitting & Sensor Fabrication

To create a knitted fabric, one or more yarns are formed into knitted loops

that loop through existing loops. With CNC knitting machines, the action of each needle and yarn carrier is controlled individually, allowing us to vary the stitch patterns, yarn materials, and geometry of a single knitted object with minimal post-processing. This gives the design freedom to create a multi-material wearable sensor that seamlessly integrates the sensor and the garment.

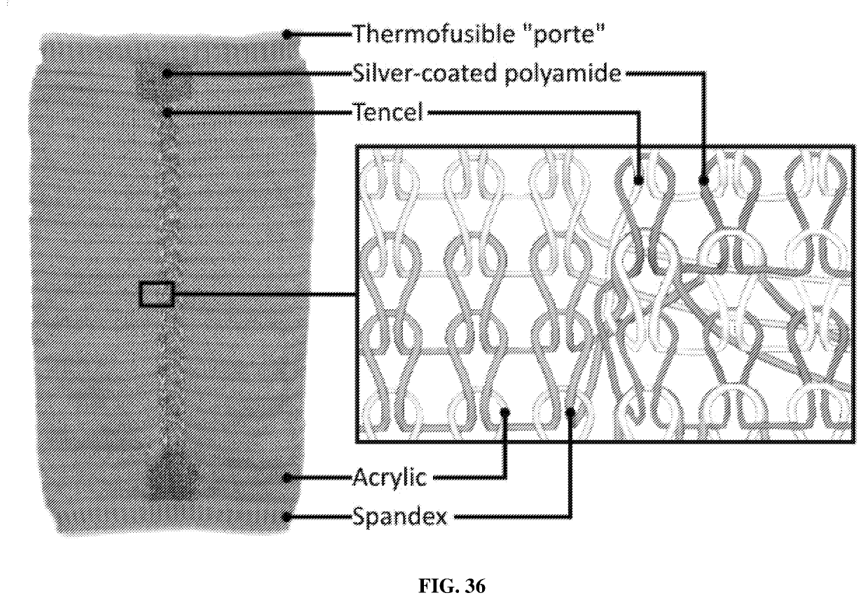

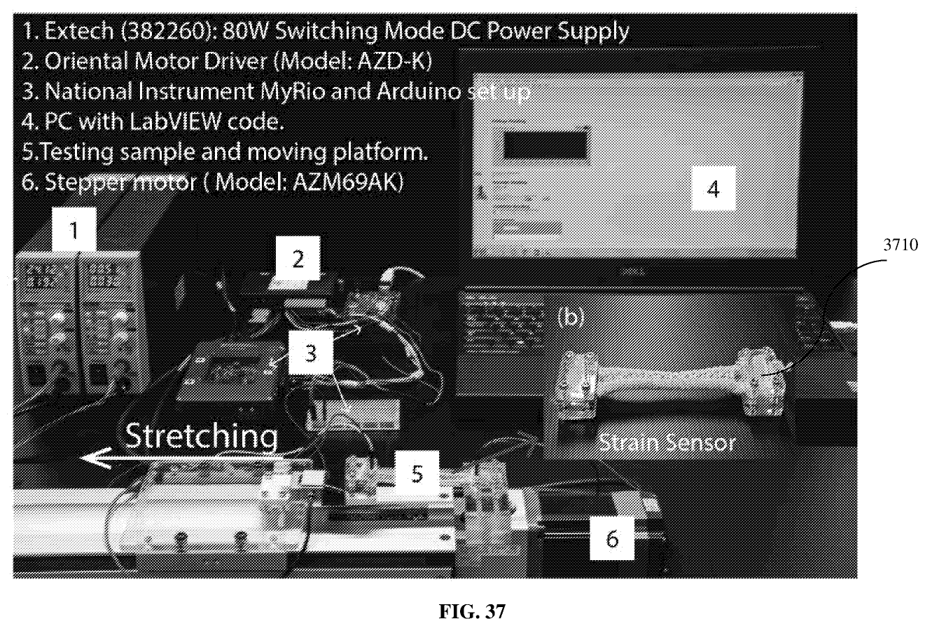

In various example experiments, knitted strain sensors that can be used to sense the bending angle of the knee is characterized. For the first set of experiments, 7 standalone sensors were knitted on a Shima Seiki MACH2XS153 WHOLEGARMENT® 15 gauge knitting machine. The sensor was knitted with alternating courses (rows) of electrically conductive silver-coated polyamide yarn (supplier: Statex, product: Shieldex® 235/36 dtex 2 ply HC+B) and non-conductive Tencel™ yarn (supplier: Lenzing A G, product: 15/1 siro spun). The sensor was knitted using purl (garter) stitch pattern and measured approximately 5×84 mm in the relaxed state. FIG. 36 shows a knitted sensor with close-up showing yarns and stitch patterns.

Since an actual wearable sensor would integrate the sensor within a garment, the sensor was knitted within a tube of single jersey (stockinette) fabric that alternated between 2 courses of non-conductive spandex covered yarn (supplier: Zhejiang Kangjiesi, product: 210D spandex with 2×75D polyester) and 2 courses of acrylic yarn (supplier: Miyama Tex, product: Guanti 2/32). To pretension the sensor and prevent it from appearing wrinkled, the non-conductive fabric was knitted with twice the number of courses as the sensor so that the sensor would be stretched by the surrounding non-conductive fabric. To make it easier to mount the sensors onto the experimental setup, the tubes were cut along the sides and the cut edges were overlocked to prevent unravelling.

### B. Experimental Setup & Sensor Characterization

The experimental setup includes two Extech (382260)-80 W switching mode DC power supply and stepper motor from Oriental motor (Model AZM69AK). The first power supply provides a constant current across the knitted sensor and the second power supply provides a constant voltage to the load cell. The stepper motor is controlled by an Arduino Uno to stretch the knitted sensor at a specified speed along a linear stage, and the measured output voltage from the knitted sensor and the encoder data from the stepper motor were simultaneously recorded at 200 Hz (5 ms) using a real-time embedded evaluation board (National Instrument: MyRio). The complete experimental setup for sensor characterization is shown in FIG. 37. The assembled strain sensor 3710 is shown in FIG. 37.

The interface for attaching the knitted sensor to the experimental setup is shown as 3710 in FIG. 37. It includes two rectangular clamps attached to the ends of the sensor. The clamps were laser cut from a 5 mm thick transparent acrylic sheet, and the knitted sensor was then secured using four socket screws and five connectivity pins to prevent the knitted sensor from sliding during the test. A small constant current of 30 mA was supplied to the sensor through the connectivity pins and the output voltage was measured across the knitted sensor as its resistance changes under strain.

All tests were conducted using the automated experimental setup and the measured output voltage and encoder data were normalised according to the initial voltage

$${ε}_{v} = ( \frac{{V}_{i} - {V}_{O}}{{V}_{O}} )$$

and length

$${ε}_{x} = ( \frac{{L}_{i} - {L}_{O}}{{L}_{O}} ) .$$

V₀ and L₀ denote the initial voltage and length of the fabric prior to strain, and Vᵢ is the voltage, and Lᵢ is the length of the fabric at particular instance of time.

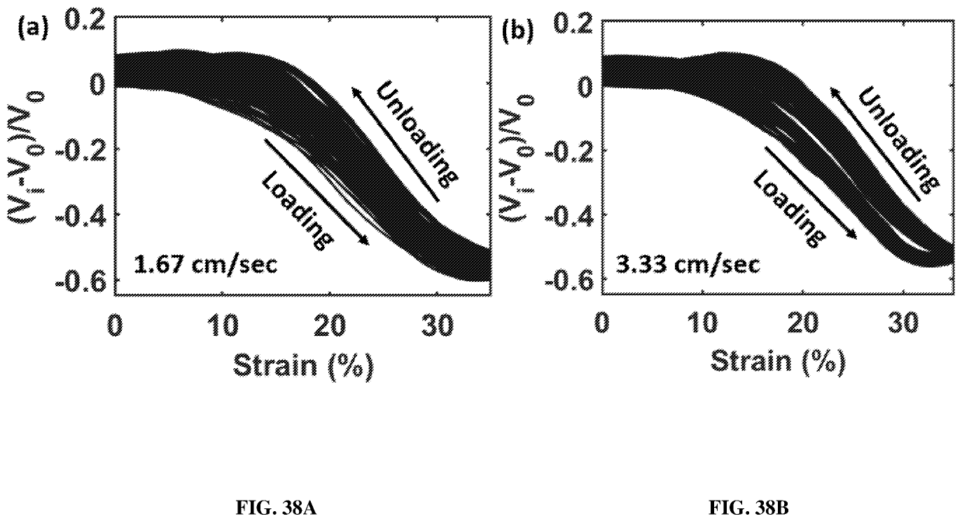

FIGS. 38A-38B show normalized voltage vs strain response of the sensor for 30 cycles of loading-unloading at 1.67 cm/sec speed, and 3.33 cm/sec speed, respectively. More particularly, FIGS. 38A-38B show the response of the sensor to cyclic loading of maximum 35% strain at two different strain rates. The sensor is stretched at a speed of 1.67 cm/sec and 3.33 cm/ sec for 30 cycles. The hysteresis loop becomes repeatable after the first few cycles. FIG. 38A shows the response of the sensor from third cycle onwards. As can be seen, the sensor shows hysteresis behaviour upon cyclic loading. FIG. 38B shows the response of the sensor when subjected to cyclic loading at 3.33 cm/sec speed.

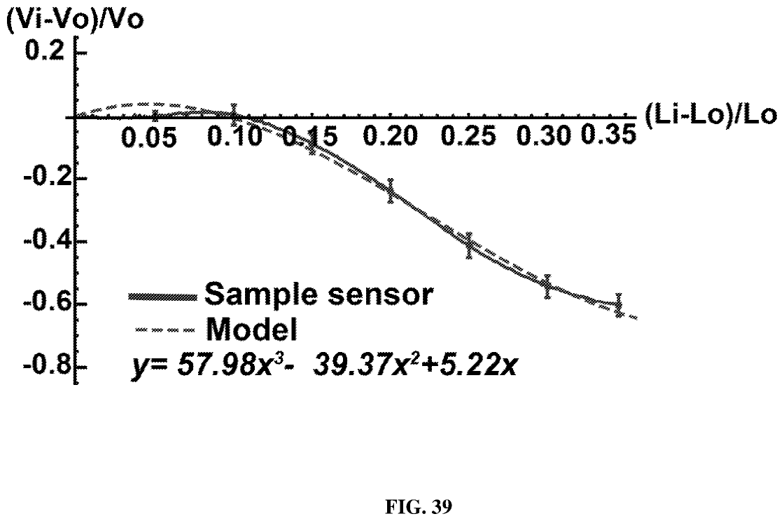

In various example embodiments, a simplified cubic polynomial model was used to fit the sensor response. FIG. 39 shows a normalized voltage-strain relationship graph. More particularly, FIG. 39 shows the response of the sensor in solid line for fifth cycle when stretched at 3.33 cm/sec. The fitted cubic polynomial model is shown in dashed line.

### Wearable Sensor Design & Knee Joint Parameterization

A customized knee brace with the characterized sensors embedded in it is provided according to various example embodiments. The design of the knitted knee brace has three sensors embedded 5 cm apart, with the left and right sensors acting as redundancy sensors to improve the accuracy of the device. The wearable knee brace was then tested on a healthy subject's left knee and the subject performed three activities: flexion-extension of the knee, walking at 1.5 km/h and jogging at 5 km/h. All three activities were performed on the AMTI treadmill for an approximate duration of 10-12 seconds.

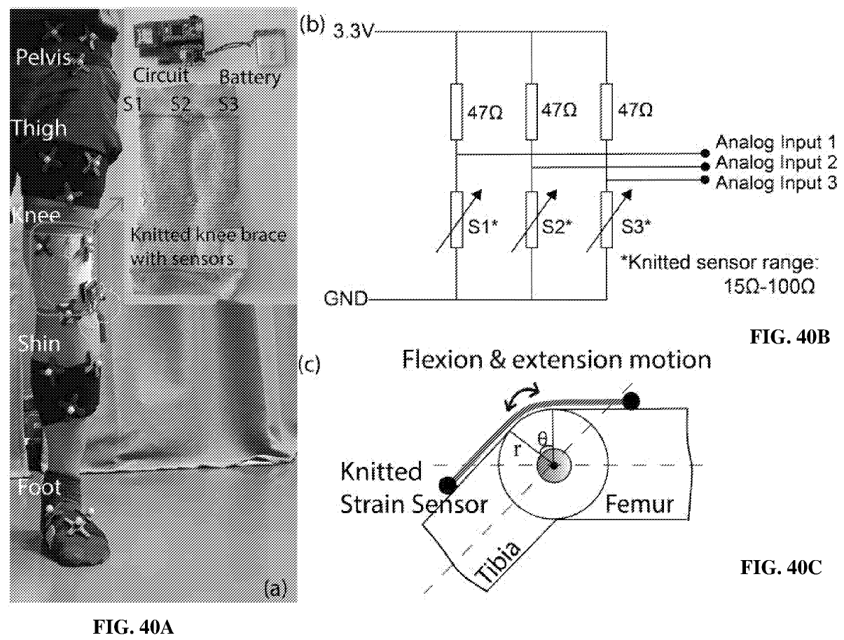

FIGS. 40A-40C show the knitted knee brace and reflective markers, electrical circuit schematic diagram and pulley model schematic diagram, respectively. The design of the electrical board and the circuit schematic diagram is shown in FIG. 40A and FIG. 40B, respectively. We used three potential dividers in parallel to read the three sensor inputs. Teensy has two 12-bit analog to digital converters, allowing us to take readings with a resolution of up to 0.8 mV on each pin. Since the voltage in every node is 3.3V, we can use the analog reading at each pin. Since the voltage in every node is 3.3V, we can use the analog reading at each pin to calculate the sensors' resistance using the following Eq.(1). The output voltage reading was recorded on a SD memory card during the three different activities.

R_{KS}=(V_{AI}×47 Ω)/(3.3V−V_{AI}) Equation (1)

To validate our knee brace measurement accuracy, we used a Vicon (Oxford, UK) system as the reference standard for comparison. A total of 15 reflective markers were attached to the subject's lower left extremity, and all three activities were recorded on nine motion-capture cameras. The motion data from the Vicon system was sampled at 100 Hz, and the data from the knitted sensors were sampled at 6 Hz. Both data sets were then processed in MATHEMATICA and the knee angle motion along the sagittal plane was compared. The coordinate locations of the 15 reflective markers were used to calculate the center of rotation (COR) and the axis based. More information on the calculation may be obtained in Tanet al., “Motion generation of passive slider multiloop wearable hand devices.” Journal of Mechanisms and Robotics 9.4 (2017), which is herein incorporated by reference in its entirety for all purposes. To relate the data collected from the electrical circuit to knee angular motion, we utilized the pulley model system shown in FIG. 40C. First, the strains were calculated using the polynomial function model. Next using the calculated strain and the subject's anthropometric radius, we obtained the knee angular motion using Eq. (2) along the sagittal plane.

L=rθ Equation (2)

### Results

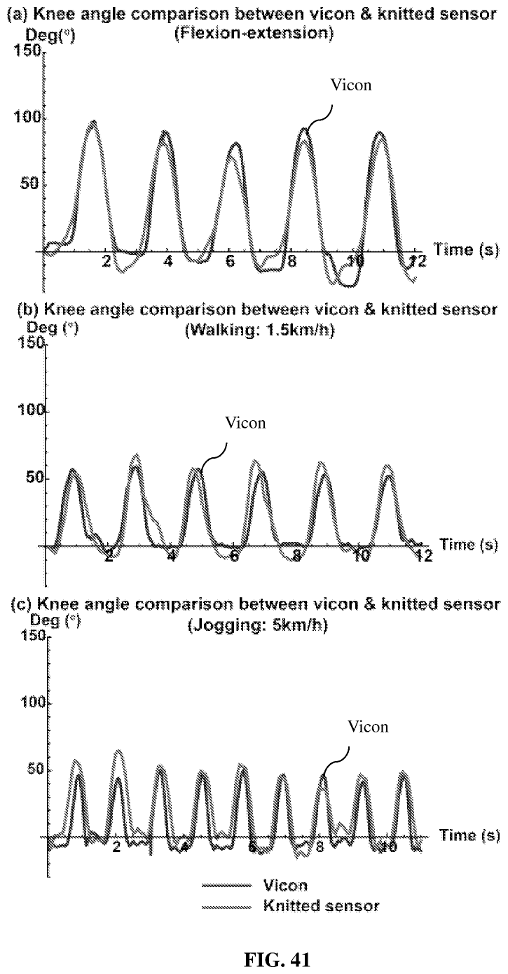

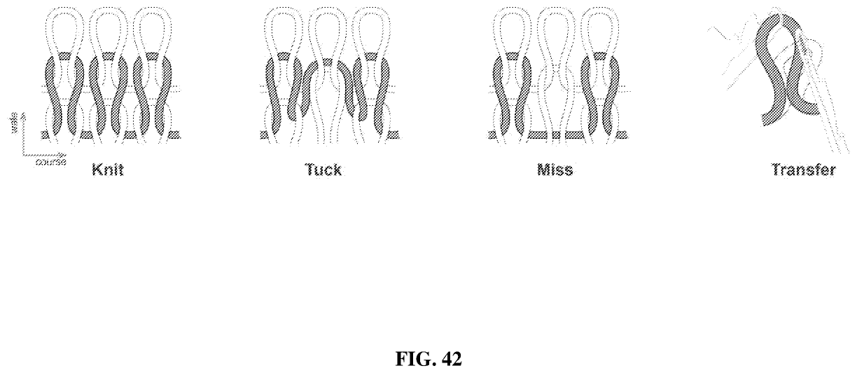

The comparisons between the reference standard Vicon and the middle knitted knee brace sensor for flexion-extension, walking and jogging are shown in FIG. 41. More particularly, FIG. 41 shows angle comparison between Vicon and middle knitted knee sensor for (a) flexion-extension, (b) walking (1.5 km/h) and (c) jogging (5 km/h). The results obtained from the three activities demonstrate that the knitted knee brace sensor can be used to monitor the knee motion along the sagittal plane. For the knee flexion-extension activity, the maximum peak error of 7.89° occurs at the peak of third cycle. In the walking gait cycles, the maximum peak error was 9.62°. for the jogging gait cycles, the maximum peak error was 16.46°. to evaluate the wearable knee brace sensor accuracy, we also calculate the Spearman's coefficient rₛ values for all three activities. Two variable are considered to share a moderate monotonic relationship if their rₛ≥0.75.

The individual rₛ values for flexion-extension, walking and jogging were 0.92, 0.84 & 0.86 respectively and the average rₛ was 0.87. These values demonstrate that there is a strong relationship between the knitted knee brace sensor and the reference standard.

The knitted knee brace thus demonstrates the ability to track the knee motion on the sagittal plane for walking and jogging activities with reasonable accuracy. However, there are still errors generated that are not entirely negligible. As can be seen, most of the error occurs during the stance phase for both walking & jogging trials. The angle difference during the stance phase is between 11.32-29.35° and these errors could be due to the following reasons. First, the electrical board's low sampling frequency results in data loss and a lower resolution for comparison. Second, the model does not account for rate-dependent hysteresis and its effect on the sensor's electromechanical property during different operating speeds. Lastly, we noticed wrinkles when the subject was wearing the knee brace. These wrinkles may create uneven contact points in the conductive fabric, resulting in a higher error during the gait stance phase.

Various example embodiments provide a method to directly integrate sensing capability into a garment. We have characterized the electromechanical property of our knitted sensor design and validated its usability by demonstrating that it can be used to track the knee motion along the sagittal plane for the three activities. Various example embodiments may improve the knitted sensor design to achieve a better working range and sensitivity and improve the model by incorporating the strain-rate hysteresis effect. At the garment design level, we can preload the sensor to prevent wrinkles and minimize errors during the stance phase in the gait cycles.

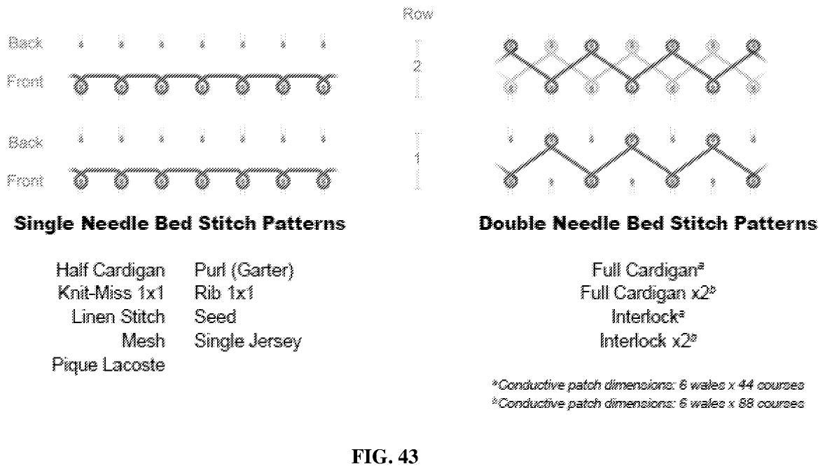

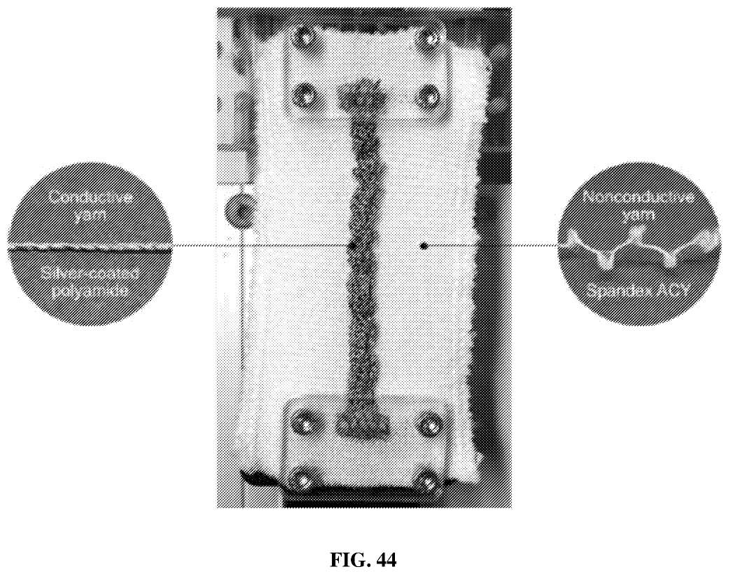

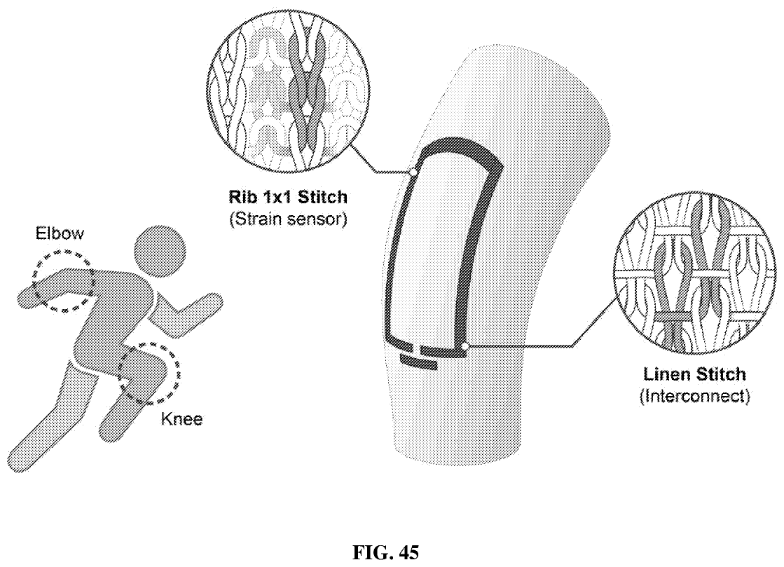

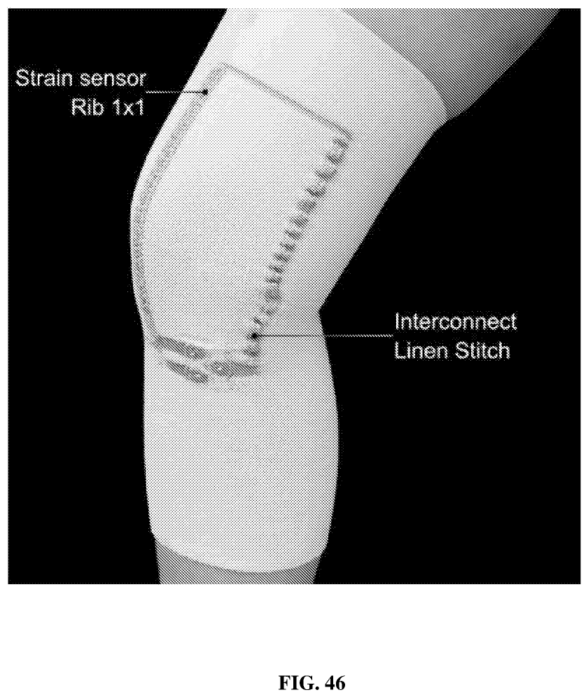

FIG. 42 shows another schematic of stitch types according to various example embodiments. FIG. 43 shows schematic of single needle bed stitch patterns and double needle bed stitch patterns according to various example embodiments. FIG. 44 shows another image illustrating a knitted conductive sample mounted on linear cyclic strain test setup and close-up diagrams of the yarn. FIG. 45 shows a schematic of another knee brace, showing the distribution of the sensor and interconnects in the wearable according to various example embodiments. FIG. 46 shows an image of another knee brace, showing the distribution of the sensor and interconnects in the wearable according to various example embodiments.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1.-42. (canceled)

43. A textile configured for strain sensing, comprising:
a strain sensing portion integrally knitted in the textile, wherein the strain sensing portion comprises:
a plurality of first groups of courses of yarn, each first group of courses of yarn comprising:
a first course of conductive yarn comprising a plurality of knitted loops;
a second course of dielectric yarn comprising a plurality of knitted loops; and
a third course of conductive yarn comprising a plurality of knitted loops,
wherein the second course is arranged in between the first and third courses and configured to have a higher elasticity than the first and third courses such that:
when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and
when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

44. The textile according to claim 43, wherein the second course is arranged in between the first and third courses and configured to have said higher elasticity than the first and third courses further such that:
when the strain sensing portion is at the relaxed state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be in contact with each other, and
when the strain sensing portion is at the stretched state, a pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be not in contact with each other.

45. The textile according to claim 44, wherein the second course is arranged in between the first and third courses and configured to have said higher elasticity than the first and third courses further such that:
when the strain sensing portion is at the relaxed state,
for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of the second course to be in contact with each other, and
for each pair of consecutive knitted loops of the plurality of knitted loops of the first course, the pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be in contact with each other, and
when the strain sensing portion is at the stretched state,
for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of the second course to be not in contact with each other, and
for each pair of consecutive knitted loops of the plurality of knitted loops of the first course, the pair of consecutive knitted loops of the plurality of knitted loops of the first course and a corresponding pair of consecutive knitted loops of the plurality of knitted loops of the third course are caused by a corresponding pair of consecutive knitted loops of the plurality of knitted loops of the second course to be not in contact with each other.

46. The textile according to claim 44, wherein
the knitted loop of the plurality of knitted loops of the first course, the corresponding knitted loop of the plurality of knitted loops of the third course, and the corresponding knitted loop of the plurality of knitted loops of the second course are along a same wale of the strain sensing portion, and
the pair of consecutive knitted loops of the plurality of knitted loops of the first course, the corresponding pair of consecutive knitted loops of the plurality of knitted loops of the third course and the corresponding pair of consecutive knitted loops of the plurality of knitted loops of the second course are along a same pair of wales of the strain sensing portion.

47. The textile according to claim 43, wherein the ratio of Young's modulus of each of the first and third courses to Young's modulus of the second course is 10³ or greater.

48. The textile according to claim 43, wherein when the strain sensing portion is at the stretched state, the strain sensing portion is stretched in a wale-wise direction.

49. The textile according to claim 43, wherein the first, second and third courses are consecutive courses.

49. The textile according to claim 43, wherein each of the first, second and third courses is formed according to a front knit-back knit stitching pattern.

50. The textile according to claim 43, wherein each intermediate first group of the plurality of first groups of courses of yarn overlaps with an immediately subsequent first group of courses of yarn and an immediately preceding first group of courses of yarn, wherein the first course of the intermediate first group is the third course of the immediately subsequent first group and the third course of yarn of the intermediate first group is the first course of the immediately preceding first group.

51. The textile according to claim 43, further comprising:
a resistor portion integrally knitted in the textile, wherein the resistor portion comprises:
a plurality of second groups of courses of yarn, each second group of courses of yarn comprising:
a first course of conductive yarn comprising a plurality of knitted loops;
a plurality of second courses of dielectric yarn, each second course comprising a plurality of knitted loops; and
a third course of conductive yarn comprising a plurality of knitted loops, wherein
knitted loops of each pair of consecutive knitted loops of the plurality of knitted loops of the first course of conductive yarn are separated therebetween by one or more wales of dielectric yarn,
knitted loops of each pair of consecutive knitted loops of the plurality of knitted loops of the third course of conductive yarn are separated therebetween by one or more wales of dielectric yarn, and
the plurality of second courses are arranged in between the first and third courses and said each second course is configured to have a higher elasticity than the first and third courses such that:
when the resistor portion is at a relaxed state, a knitted loop of the plurality of knitted loops of the first course and a corresponding knitted loop of the plurality of knitted loops of the third course are caused by a corresponding knitted loop of the plurality of knitted loops of said each second course to be not in contact with each other, and
when the resistor portion is at a stretched state, a knitted loop of the plurality of knitted loops of the first course and a corresponding knitted loop of the plurality of knitted loops of the third course are caused by a corresponding knitted loop of the plurality of knitted loops of said each second course to be not in contact with each other.

52. The textile according to claim 51, wherein the plurality of second courses are arranged in between the first and third courses and said each second course is configured to have a higher elasticity than the first and third courses such that:
when the resistor portion is at the relaxed state,
for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of said each second course to be not in contact with each other, and
when the resistor portion is at the stretched state,
for each knitted loop of the plurality of knitted loops of the first course, the knitted loop of the plurality of knitted loops of the first course and a corresponding knitted loop of the plurality of knitted loops of the third course are caused by a corresponding knitted loop of the plurality of knitted loops of said each second course to be not in contact with each other.

53. The textile according to claim 51, wherein
the knitted loop of the plurality of knitted loops of the first course, the corresponding knitted loop of the plurality of knitted loops of the third course, and the corresponding knitted loop of the plurality of knitted loops of said each second course are along a same wale of the resistor portion.

54. The textile according to claim 51, wherein the ratio of Young's modulus of each of the first and third courses to Young's modulus of each of the plurality of second courses is 10³ or greater.

55. The textile according to claim 51, wherein when the resistor portion is at the stretched state, the resistor portion is stretched in a wale-wise direction.

56. The textile according to claim 51, wherein the first course, the plurality of second courses and the third course are consecutive courses.

57. The textile according to claim 51, wherein
each of the first and third courses is formed according to a knit-miss-knit-miss with transfer stitching pattern.

58. The textile according to claim 51, wherein each intermediate second group of the plurality of second groups of courses of yarn overlaps with an immediately subsequent second group of courses of yarn and an immediately preceding second group of courses of yarn, wherein the first course of the intermediate second group is the third course of the immediately subsequent second group and the third course of yarn of the intermediate second group is the first course of the immediately preceding second group.

59. The textile according to claim 51, further comprising:
a first interconnect portion integrally knitted in the textile, wherein the first interconnect portion comprises:
a plurality of consecutive courses of conductive yarn, each course of conductive yarn comprising a plurality of knitted loops formed according to a knit-miss stitching pattern.

60. The textile according to claim 59, further comprising:
a second interconnect portion integrally knitted in the textile, wherein the second interconnect portion comprises:
a plurality of consecutive courses of conductive yarn, each course of conductive yarn comprising a plurality of knitted loops formed according to a knit-miss with transfer stitching pattern.

61. The textile according to claim 60, wherein the strain sensing portion and the resistor portion are communicatively coupled based on at least one of the first and second interconnect portions to form a circuit for measuring strain on the strain sensing portion based on changes in resistance of the strain sensing portion detected in response to the strain on the strain sensing portion.

62. A knitted wearable configured to be worn by a subject for motion sensing of the subject, the knitted wearable comprising a textile configured for strain sensing, the textile comprising:
a strain sensing portion integrally knitted in the textile, wherein the strain sensing portion comprises:
a plurality of first groups of courses of yarn, each first group of courses of yarn comprising:
a first course of conductive yarn comprising a plurality of knitted loops;
a second course of dielectric yarn comprising a plurality of knitted loops; and
a third course of conductive yarn comprising a plurality of knitted loops,
wherein the second course is arranged in between the first and third courses and configured to have a higher elasticity than the first and third courses such that:
when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and
when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

63. A method of manufacturing a textile for strain sensing, the method comprising:
forming a strain sensing portion integrally knitted in the textile, wherein forming the strain sensing portion comprises:
knitting a plurality of first groups of courses of yarn, each first group of courses of yarn comprising:
a first course of conductive yarn comprising a plurality of knitted loops;
a second course of dielectric yarn comprising a plurality of knitted loops; and
a third course of conductive yarn comprising a plurality of knitted loops,
wherein the second course is arranged in between the first and third courses and configured to have a higher elasticity than the first and third courses such that:
when the strain sensing portion is at a relaxed state, a knitted loop of the plurality of knitted loops of the first course and a corresponding knitted loop of the plurality of knitted loops of the third course are caused by a corresponding knitted loop of the plurality of knitted loops of the second course to be in contact with each other, and
when the strain sensing portion is at a stretched state, a knitted loop of the plurality of knitted loops of the first course and a corresponding knitted loop of the plurality of knitted loops of the third course are caused by a corresponding knitted loop of the plurality of knitted loops of the second course to be not in contact with each other.

64. The method according to claim 63, further comprising:
forming a resistor portion integrally knitted in the textile, wherein forming the resistor portion comprises:
knitting a plurality of second groups of courses of yarn, each second group of courses of yarn comprising:
a first course of conductive yarn comprising a plurality of knitted loops;
a plurality of second courses of dielectric yarn, each second course comprising a plurality of knitted loops; and
a third course of conductive yarn comprising a plurality of knitted loops, wherein
knitted loops of each pair of consecutive knitted loops of the plurality of knitted loops of the first course of conductive yarn are separated therebetween by one or more wales of dielectric yarn,
knitted loops of each pair of consecutive knitted loops of the plurality of knitted loops of the third course of conductive yarn are separated therebetween by one or more wales of dielectric yarn, and
the plurality of second courses are arranged in between the first and third courses and said each second course is configured to have a higher elasticity than the first and third courses such that:
when the resistor portion is at a relaxed state, a knitted loop of the plurality of knitted loops of the first course and a corresponding knitted loop of the plurality of knitted loops of the third course are caused by a corresponding knitted loop of the plurality of knitted loops of said each second course to be not in contact with each other, and
when the resistor portion is at a stretched state, a knitted loop of the plurality of knitted loops of the first course and a corresponding knitted loop of the plurality of knitted loops of the third course are caused by a corresponding knitted loop of the plurality of knitted loops of said each second course to be not in contact with each other.
